(19) United States
(12) Patent Application Publication
(10) Pub. No.: US 2009/0311334 A1
(43) Pub. Date: Dec. 17, 2009

(54) MUCOSAL IMMUNOGENIC SUBSTANCES COMPRISING A POLYINOSINIC ACID - POLYCYTIDILIC ACID BASED ADJUVANT

(76) Inventors: Hiaxiang LIN, Beijing (CN); Lie Tao Victor Li, Singapore (SG)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/160,584

(22) PCT Filed: Jun. 27, 2006
(86) PCT No.: PCT/SG2006/000177

§ 371 Date: Nov. 12, 2008

(30) Foreign Application Priority Data
Jan. 13, 2006 (US) 11331839

(51) Int. Cl. A61K 9/16; A61K 39/00; A61K 9/12

(52) U.S. Cl. 424/490; 424/184.1; 424/45

(57) ABSTRACT

The present invention provides a polynucleotide adjuvant (PICKCa) composition and methods of use in eliciting an immune response, in particular a mucosal immune response. The polynucleotide adjuvant comprises of a polyriboinosinic-polyri-bocytidylic acid (PIC), at least one antibiotic and at least one positive ion. The present invention also provides an immunogenic composition comprising the polynucleotide adjuvant composition together with other immunogenic compositions such as an antigen (e.g., as in a vaccine). The present invention further contemplates methods of use of such adjuvant compositions, particularly in eliciting an immune response, in particular a mucosal immune response to an antigenic compound.

### FIELD OF INVENTION

The invention generally relates to immunogenic compositions and methods of their use. More specifically the invention relates to an immunogenic composition comprising a polynucleotide adjuvant in combination with one or more antigenic substances to be used to elicit disease specific mucosal immune response in a host.

### BACKGROUND OF INVENTION

The immune system may exhibit both specific and nonspecific immunity. Nonspecific immunity encompasses various cells and mechanisms such as phagocytosis (the engulfing of foreign particles or antigens) by macrophages or granulocytes, and natural killer (NK) cell activity, among others. Nonspecific immunity relies on mechanisms less evolutionarily advanced and does not display the acquired nature of specificity and memory, which are exemplary hallmarks of a specific immune response. The key differences between specific and nonspecific immunity are based upon B and T cell specificity. These cells predominantly acquire their responsiveness after activation with a specific antigen and have mechanisms to display memory in the event of future exposure to that specific antigen. As a result, vaccination (involving specificity and memory) is an effective protocol to protect against harmful pathogens.

Generally, B and T lymphocytes, which display specific receptors on their cell surface for a given antigen, produce specific immunity. The specific immune system may respond to different antigens in two ways: 1) humoral-mediated immunity, which includes B cell stimulation and production of antibodies or immunoglobulins and helper T cells (predominantly Th2), and 2) cell-mediated immunity, which generally involves T cells including cytotoxic T lymphocytes (CTLs), although other cells are also involved in the generation of a CTL response (e.g., antigen presenting cells and Th1cells).

The immune system has developed a distinct and specialized repertoire of immune responses to combat infections. The human immune system may be broadly sub-divided into two interacting sub-systems. The systemic immune system, comprising the lymph nodes, bone marrow and spleen, that patrols the inner organs and tissues, and the mucosal immune system comprising the lymphoid tissues associated with mucosal surfaces and external secretory glands which provides a defensive barrier against pathogens entering the body through epithelial lining of respiratory, gastrointestinal, sensory and genitourinary tracts.

Immune responses of the systemic and mucosal immune system have evolved with specific functions and largely remain distinct in their defensive mechanisms against pathogens. Mucosal immunity for instance is generally characterized by the presence of a specialized class of antibodies, immunoglobulin A (IgA) antibodies, primarily secretory IgA (S-IgA) protecting the mucosal surfaces. S-IgA antibodies neutralize pathogens in the mucosae that have not yet crossed the mucosal barrier.

In general, existing immunization strategies which involve intramuscular, subcutaneous, intraperitoneal or intradermal administration of antigens evoke the systemic immune system in the production of different classes of antibodies for instance, immunoglobulin G (IgG) that neutralize pathogens after they have entered the body. Vaccines administered by injection tend not evoke substantial S-IgA response. Furthermore, systemic immunity does not necessarily provide for inhibition of the entry of pathogens into the body via the mucosal surfaces. Thus a vaccination strategy that only induces a systemic immune response leaves the subject prone to infection via the mucosal surface with the body's immune system fighting the pathogen once it is in circulation.

Mucosal administration on the other hand induces mucosal (at local and sometimes remote sites of administration) and systemic immune responses. Furthermore, traditional methods of injected immunization regimes are known to have a number of drawbacks, including risk of infection and low tolerance by many individuals with cases of induration (hardening of tissue), hemorrhage (bleeding) and/or necrosis (local death of tissue) at the injection site.

However it is not possible to conclude that since an adjuvant enhances a systemic immune response it will necessarily also enhance a mucosal immune response. A typical example is aluminum hydroxide which enhances the systemic immunogenicity of a substance on intramuscular, subcutaneous, intraperitoneal or intradermal administration but is ineffective in enhancing a mucosal immune response when administered by injection or by a mucosal route.

There has been an intensive search in recent years for novel adjuvants, including those to enhance a mucosal immune response. Efforts to take advantage of S-IgA protection at mucosal barriers have included oral immunization, as well as applying monoclonal S-IgA antibodies directly to respiratory surfaces in an effort to protect against pathogen entry. However there remains a medical need for safe and effective adjuvants that are able to elicit a beneficial mucosal immune response in a host.

The present invention provides novel immunogenic compositions that exhibit improved safety and efficacy profiles; and methods of use of such compositions to enhance a mucosal immune response. Subject immunogenic compositions include a polynucleotide adjuvant and an antigen.

Literature

The following references may be of interest:
JP 1093540A2;
U.S. Pat. No. 4,124,702
U.S. Pat. No. 3,692,899
U.S. Pat. No. 3,906,092
U.S. Pat. No. 4,389,395
U.S. Pat. No. 4,349,538
U.S. Pat. No. 4,024,241
U.S. Pat. No. 3,952,097
Houston et al., Infection and Immunity, 14:318-9, 1976C
Wright and Adler-Moore, Biochemical and Biophysical Research Communications, 131: 949-45, 1985
Lin, et al., A new immunostimulatory complex (PICKCa) in experimental rabies: antiviral and adjuvant effects, Arch Virol, 131: 307-19, 1993
Chinese Patent 93105862.7
Gupta R. K. et al., Adjuvants—a balance between toxicity and adjuvanticity, Vaccine, 11:293-306, 1993
Arnon, R. (Ed.) Synthetic Vaccines 1:83-92, CRC Press, Inc., Boca Raton, Fla., 1987
Sela, M., Science 166:1365-1374 (1969)
U.S. Pat. No. 6,008,200
Ellouz et al., Biochem. & Biophy. Res. Comm., 59:1317, 1974
U.S. Pat. No. 4,094,971
U.S. Pat. No. 4,101,536
U.S. Pat. No. 4,153,684
U.S. Pat. No. 4,235,771
U.S. Pat. No. 4,323,559
U.S. Pat. No. 4,327,085
U.S. Pat. No. 4,185,089
U.S. Pat. No. 4,082,736
U.S. Pat. No. 4,369,178
U.S. Pat. No. 4,314,998
U.S. Pat. No. 4,082,735
U.S. Pat. No. 4,186,194
U.S. Pat. No. 6,468,558
New Trends and Developments in Vaccines, edited by Voller et al., University Park Press, Baltimore, Md., USA, 1978
Klein, J., et al., Immunology (2nd), Blackwell Science Inc., Boston (1997)
Gupa R. K. and Siber G. R., Adjuvants for human vaccines—current status, problems and future prospects, Vaccine, 13 (14): 1263-1276, 1995
Richard T Kenney et al. Meeting Report—2^{nd} meeting on novel adjuvants currently in/close to human clinical testing, Vaccine 20 2155-2163, 2002
Laboratory Techniques in Rabies Edited by F X Meslin, M M Kaplan, H Koprowski 4^{th},1996, Edition ISBN 92 4 1544 1

### SUMMARY OF THE INVENTION

In general, the present invention, relates to immunogenic compositions comprising a polyinosinic acid-polycytidylic acid, kanamycin and calcium complex adjuvant and their methods of use to elicit a disease specific mucosal immune response.

Accordingly, there is provided an immunogenic composition comprising: (a) a polynucleotide adjuvant comprising: a polyriboinosinic-polyribocytidylic acid (PIC), at least one an antibiotic, and at least one positive ion; and (b) at least one antigen; wherein the composition is formulated for mucosal administration.

Particularly, the invention relates to the application of immunogenic compositions comprising a polyinosinic acid-polycytidylic acid, kanamycin and calcium complex as an adjuvant that is safe for use in humans and non-human animals, which when administered in combination with antigenic and/or immunomodulating substance(s), enhances the specific mucosal immune response and in certain applications enhances both a specific mucosal and systemic immune response.

More in particular, the immunogenic composition according to the invention may comprise a polynucleotide adjuvant composition molecules heterogeneous for molecular weight, wherein the molecular weight is at least 66,000 Daltons.

### BRIEF DESCRIPTION OF THE DRAWINGS

FIG. 1—ELISA detection of specific S-IGA titers in lung supernatant after immunization with vaccines comprising PIKA and/or whole inactivated SARS antigen

FIG. 2—ELISA detection of specific IgA titers in blood serum after immunization with vaccines comprising PIKA and/or whole inactivated SARS antigen

FIG. 3—ELISA detection of specific IgG titers in blood serum after immunization with vaccines comprising PIKA and/or whole inactivated SARS antigen

FIG. 4—ELISA detection of specific S-IgA titers in lung supernatant after immunization with vaccines comprising PIKA and/or inactivated split influenza antigen

FIG. 5—ELISA detection of specific S-IgA titers in intestinal supernatant after immunization with vaccines comprising PIKA and/or inactivated split influenza antigen

FIG. 6—ELISA detection of specific IgG titers in blood serum after immunization with vaccines comprising PIKA and/or inactivated split influenza antigen

FIG. 7—ELISA detection of specific IgA titers in blood serum after immunization with vaccines comprising PIKA and/or inactivated split influenza antigen

FIG. 8—ELISPOT detection of murine splenocytes producing IL-2 after immunization with vaccines comprising PIKA and/or inactivated split influenza antigen

FIG. 9: ELISA detection of specific S-IgA in lung supernatant (32× dilution) after immunization with vaccines comprising PIKA or Al(OH)3 and/or split inactivated influenza antigen

FIG. 10: ELISA detection of specific S-IgA in intestine supernatant (32× dilution) after immunization with vaccines comprising PIKA or Al(OH)3 and/or split inactivated influenza antigen

FIG. 11: ELISPOT detection of murine splenocytes producing IFN-gamma after immunization with vaccines comprising PIKA or alum and/or split inactivated flu antigen

FIG. 12: ELISPOT detection of murine splenocytes producing IL-2 after immunization with vaccines comprising PIKA or alum and/or split inactivated flu antigen

### DETAILED DESCRIPTION OF EXEMPLARY EMBODIMENTS OF THE INVENTION

The present invention may be understood more readily by reference to the following detailed description of certain embodiments of the invention and the Examples included herein.

Throughout this application, where publications are referenced, the disclosures of these publications are hereby incorporated by reference, in their entireties, into this application in order to describe more fully the state of art to which this invention pertains.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “an immunogenic composition” includes a plurality of such compositions and reference to “the antigen” includes reference to one or more antigens and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### Definitions of Terms

Prior to setting forth details of the present invention it may be useful to an understanding thereof to set forth definitions of several terms that are used herein.

The term “adjuvant,” as used herein, refers to any substance or mixture of substances that increases or diversifies the immune response of a host to an antigenic compound. Specifically:
1. The term “PICKCa” generally refers to a composition of poly I:C, kanamycin and calcium irrespective of particular physical and immunogenic properties.
2. “Av-PICKCa” refers to a form of PICKCa used commercially as an antiviral drug.
3. “PIKA” refers to a composition of the invention comprising poly I:C, an antibiotic (e.g., kanamycin), and a positive ion (e.g., calcium), where the PIKA is characterized by physical characteristics (e.g., molecular weight, size, and the like) such that upon administration, PIKA exhibits characteristics of an adjuvant with reduced adverse side effects (e g., reduced toxicity) relative to, for example, PICKCa and greater potency (e.g., stimulates an enhanced immune response) relative to, for example, Av-PICKCa.

The term “Poly I:C” or “PIC” refers to a composition comprising polyriboinosinic and polyribocytidylic nucleic acids, which may also be referred to as polyinosinic acid-polycytidylic acid, respectively.

“PIC-containing molecule” or “PIC-containing compound” refers to, without limitation, PIC, which may be optionally complexed or otherwise combined with at least one or both of an antibiotic (e.g., kanamycin) and a positive ion (e.g., calcium) present in a composition comprising the PIC-containing molecule. In one embodiment, the PIC-containing molecule does not include poly-L-lysine or a derivative thereof in the complex.

“Heterogeneous” as used herein in the context of the adjuvant compositions of the invention indicates that components of the composition, e.g., the PIC-containing molecules, are not uniform with respect to a physical characteristic of molecular weight, size, or both. Where a composition is described as heterogenous for a given physical characteristic, and is further described by a range of values for that physical characteristic, the composition is said to be composed substantially of molecules characterized by molecules having a physical characteristic that is distributed within and across the recited range. While the composition may not contain a molecule representative of every physical characteristic value within the upper and lower limits of a recited range, the composition will generally include at least one molecule having the physical characteristic of the upper value and of the lower value. The composition in certain embodiments may include molecules outside the stated range of physical characteristics used to describe the composition. The molecules that are present in the composition outside the prescribed range do not materially affect the basic and novel characteristics of the composition.

The term “mucosal” or “mucosal membrane” or “mucosal surface” refers to the surfaces, passages and cavities that are in contact directly or indirectly with the exterior environment, including the surfaces of the respiratory, digestive, sensory and genitourinary systems. “Mucosal surface of the gastrointestinal tract” is meant to include mucosa of the bowel (including the small intestine and large intestine), rectum, stomach (gastric) lining, oral cavity, and the like.

The term “formulated for mucosal administration” refers to a composition that is adapted for and thus compatible with administration to the mucosa (e.g., to a mucosal surface or mucosal membrane). In some embodiments, the composition is formulated for mucosal administration by a route other than rectal, vaginal, nasal, oral, or opthamalic (e.g., the composition is formulated for administration to lung tissue, e.g., by pulmonary administration.

The term “individual,” used interchangeably herein with “host,” “subject,” and “animal,” includes humans and all domestic e.g. livestock and pets and wild mammals and fowl, including, without limitation, cattle, horses, cows, swine, sheep, goats, dogs, cats, rabbits, deer, mink, chickens, ducks, geese, turkeys, game hens, and the like.

The term “antibody” includes polyclonal and monoclonal antibodies, as well as antigenic compound binding fragments of such antibodies including Fab, F(ab′)2, Fd, Fv fragments, and single chain derivatives of the same. In addition, the term “antibody” includes naturally occurring antibodies as well as non-naturally occurring antibodies, including, for example, chimeric, bifunctional and humanized antibodies, and related synthetic isoforms. The term “antibody” is used interchangeably with “immunoglobulin.”

As used herein, the term “antigenic compound” refers to any substance that can be recognized by the immune system (e.g., bound by an antibody or processed so as to elicit a cellular immune response) under appropriate conditions.

An “antigen” refers to a substance, including compositions in the form of a vaccine where the vaccine itself comprises an antigenic compound and may or may not comprise an adjuvant other than PIKA, which when administered by an appropriate route (e.g., parenterally), induces a specific immune response, for example, the formation of antibodies, including antibodies that specifically bind the antigen. Two of the characteristic features of antigens are their immunogenicity, that is, their capacity to induce a specific immune response in vivo, and their antigenicity, that is their capacity to be selectively recognized by the antibodies whose origins are the antigens.

An “antigen” as used herein includes but is not limited to cells; cell extracts; proteins; lipoproteins; glycoproteins; nucleoproteins; polypeptides; peptides; polysaccharides; polysaccharide conjugates; peptide mimics of polysaccharides; lipids; glycolipids; carbohydrates; viruses; viral extracts; bacteria; bacterial extracts; fungi; fungal extracts; multicellular organisms such as parasites; and allergens. Antigens may be exogenous (e.g., from a source other than the individual to whom the antigen is administered, e.g., from a different species) or endogenous (e.g., originating from within the host, e.g., a diseased element of body, a cancer antigen, a virus infected cell producing antigen, and the like). Antigens may be native (e.g., naturally-occurring); synthetic; or recombinant. Antigens include crude extracts; whole cells; and purified antigens, where “purified” indicates that the antigen is in a form that is enriched relative to the environment in which the antigen normally occurs and/or relative to the crude extract, for example, a cultured form of the antigen.

An “immunogenic composition” as used here in refers to a combination of two or more substances (e.g., an antigen and an adjuvant) that together elicit an immune response when administered to a host.

The term “polypeptide”, “peptide,” “oligopeptide,” and “protein”, are used interchangeably herein, and refer to a polymeric form of amino acids of any length, which can include coded and non-coded amino acids, chemically or biochemically modified or derivatized amino acids, and polypeptides having modified peptide backbones.

An “effective amount of an antigenic compound” refers to an amount of antigenic compound which, in optional combination with an adjuvant, will cause the subject to produce a specific immunological response to the antigenic compound.

The term “immune response” refers to any response to an antigenic compound or immunogenic compound by the immune system of a vertebrate subject. Exemplary immune responses include, but are not limited to local and systemic cellular as well as humoral immunity, such as cytotoxic T lymphocytes (CTL) responses, including antigen-specific induction of CD8+CTLs, helper T-cell responses including T-cell proliferative responses and cytokine release, and B-cell responses including antibody response.

The term “eliciting an immune response” is used herein generally to encompass induction and/or potentiation of an immune response.

The term “inducing an immune response” refers to an immune response that is, stimulated, initiated, or induced.

The term “potentiating an immune response” refers to a pre-existing immune response that is improved, furthered, supplemented, amplified, enhanced, increased or prolonged.

The expression “enhanced immune response” or similar means that the immune response is elevated, improved or enhanced to the benefit of the host relative to the prior immune response status, for example, before the administration of an immunogenic composition of the invention.

The terms “mucosal immune response” and “mucosal immunity” are terms well understood in the art, and refers to an immune response characterized, at least in part, by production of secretory IgA and/or stimulation of a mucosal CTL response in mucosal tissues such as gastrointestinal tract tissues, including rectal tissues; vaginal tissues; and tissues of the respiratory tract.

The terms “humoral immunity” and “humoral immune response” refer to the form of immunity in which antibody molecules are produced in response to antigenic stimulation.

The terms “cell-mediated immunity” and “cell-mediated immune response” are meant to refer to the immunological defense provided by lymphocytes, such as that defense provided by T cell lymphocytes when they come into close proximity to their victim cells. A cell-mediated immune response normally includes lymphocyte proliferation. When “lymphocyte proliferation” is measured, the ability of lymphocytes to proliferate in response to a specific antigen is measured. Lymphocyte proliferation is meant to refer to B cell, T-helper cell or CTL cell proliferation.

The term “immunogenic amount” refers to an amount of antigenic compound sufficient to stimulate an immune response, when administered with a subject immunogenic composition, as compared with the immune response elicited by the antigen in the absence of the polynucleotide adjuvant.

The term “immunopotentiating amount” refers to the amount of the adjuvant needed to effect an increase in antibody titer and/or cell-mediated immunity when administered with an antigenic compound in a composition of the invention, as compared with the increase in antibody and/or cell mediated immunity level observed in the absence of the polynucleotide adjuvant.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, e.g., arresting its development; or relieving the disease symptom, i.e., causing regression of the disease or symptom (c) reduction of a level of a product produced by the infectious agent of a disease (e.g., a toxin, an antigen, and the like); and (d) reducing an undesired physiological response to the infectious agent of a disease (e.g., fever, tissue edema, and the like).

As used herein, the term “mixing” includes any method to combine the components of the composition; such methods include, but are not limited to, blending, dispensing, dissolving, emulsifying, coagulating, suspending, or otherwise physically combining the components of the composition.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-clilorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “unit dosage form” as used herein refers to physically discrete units suitable as unitary dosages for human and animal subjects, each unit containing a predetermined quantity of compounds of the present invention calculated in an amount sufficient to produce the desired effect in association with a pharmaceutically/physiologically acceptable diluent, carrier or vehicle.

### Exemplary Embodiments of the Invention

The present invention is directed to immunogenic compositions and methods useful for induction and/or enhancement of an immune response, which may be mucosal and/or systemic, humoral and/or cell-mediated, in a human, in a non-human animal, or in cell culture. In general, a immunogenic composition according to the invention comprises an antigen (an “antigenic composition”) and an adjuvant. The presence of the adjuvant enhances or modifies the immune response to the antigen. The adjuvant may alter the quality of the immune response by affecting the subclasses (isotypes) of immunoglobulins and /or chemokines and/or cytokines produced. As a result the innate immunity, humoral and/or cell-mediated immune responses are more effective with the presence of the adjuvant.

A particular advantage is the effectiveness of the PIKA adjuvant in combination with an antigenic substance in inducing a specific humoral immune response thereby enhancing protective immunity.

A further important advantage is that the PIKA adjuvant in combination with an antigen can induce a specific cell mediated immune response that is essential for a therapeutic vaccine for limiting and treating intracellular viral, bacterial and parasite infections.

Accordingly, included in the invention are compositions having the unique product attributes that make them most suitable for use as vaccines to be administered to animals and/or humans that address the need for a safe adjuvant, which elicits a beneficial immune response.

Accordingly, the present invention provides an adjuvant and an immunogenic composition that can be used safely in humans and animals.

Accordingly, there is provided an immunogenic composition comprising: (a) a polynucleotide adjuvant comprising: a polyriboinosinic-polyribocytidylic acid (PIC), at least one an antibiotic, and at least one positive ion; and (b) at least one antigen; wherein the composition is formulated for mucosal administration.

In particular, the immunogenic composition according to the invention may comprise a polynucleotide adjuvant composition molecules heterogeneous for molecular weight, wherein the molecular weight is at least 66,000 Daltons. The value of 66,000 Daltons corresponds to the size of about 6.4 Svedbergs. Accordingly, a molecular weight range of 66,000 to 1,200,000 Daltons corresponds to the size from about 6.4 to 24.0 Svedbergs.

More specifically, the present invention provides the PIKA adjuvant composition comprising a polynucleotide, an antibiotic and a positive ion, wherein the polynucleotide may be polyriboinosinic-polyribocytidylic acid (PIC); the antibiotic may be kanamycin, and the ion may be calcium.

In one aspect of particular interest, the invention provides for an immunogenic composition for enhancing the antigenicity of an antigenic compound comprising the polynucleotide adjuvant composition that is capable of eliciting an antigen specific cell mediated immune response.

In one aspect of particular interest, the invention provides for an immunogenic composition for enhancing the antigenicity of an antigenic compound comprising the polynucleotide adjuvant composition that is capable of eliciting an antigen specific humoral immune response.

In one aspect of particular interest, the invention provides for an immunogenic composition for enhancing the antigenicity of an antigenic compound comprising the polynucleotide adjuvant composition that is capable of eliciting a combined specific cell mediated and humoral immune response

In one aspect of particular interest, the invention provides for an adjuvant composition or immunogenic composition comprising an adjuvant composition wherein the adjuvant composition or the immunogenic composition is freeze-dried.

In one aspect of particular interest, the invention provides for the use of a polynucleotide adjuvant composition for the preparation of a medicament for enhancing the immunogenic response of a host.

Polynucleotide Adjuvant

A subject immunogenic composition comprises a PIC-containing polynucleotide adjuvant, e.g., a PIKA composition, is generally composed of polyinosinic acid, polycytidylic acid, an antibiotic (e.g., kanamycin), and a divalent cation (e.g., calcium).It will be understood that reference to PIKA herein is exemplary of such PIC-containing adjuvants.

PIC-containing adjuvants of interest can be manufactured using methods available in the art. The PIC-containing adjuvant composition can be manufactured through any appropriate process. For example the polynucleotide adjuvant composition can be manufactured by mixing of polyinosinic acid, polycytidylic acid, an antibiotic and the source of a positive ion in a sodium chloride/phosphate buffer solution that has a pH between pH6 and pH8. The polyinosinic acid and polycytidylic acid are generally provided at a concentration of 0.1 to 10 mg/ml, 0.5 to 5 mg/ml, or 0.5 to 2.5 mg/ml. The hyperchromicity value should be at least or greater than 10%, greater than 15%, greater than 20%, or greater than $50%. The preparation of the PIC and the combination with the antibiotic (e.g., kanamycin) and the positive ion (e.g., calcium) is generally conducted under quality standards consistent with international Good Manufacturing Process.

In certain embodiments of the present invention, the antibiotic component of the adjuvant is kanamycin. Where the antibiotic is kanamycin, in some embodiments, the kanamycin in the polynucleotide adjuvant composition is used together with or substituted by one or more antibiotics selected from the group including tobramycin, anthracyclines, butirosin sulfate, gentamicins, hygromycin, amikacin, dibekacin, nebramycin, metrzamide, neomycin, puromycin, streptomycin and streptozocin. The antibiotic (e.g., Kanamycin or the like) in the polynucleotide adjuvant composition of the invention is generally provided at a concentration of from about 10 units/ml to 100,000 units/ml, from about 100 units/ml to 10,000 units/ml, or from about 500 units/ml to 5,000 units/ml.

In certain embodiments of the present invention, the polynucleotide adjuvant composition further comprises a positive ion (cation), usually a divalent cation, normally a cation of an alkali metal. The positive ion is generally provided in the composition of the invention as a source of positive ions such as a salt or complex, e.g., an organic or inorganic salt or complex, usually an inorganic salt or organic complex. Exemplary positive ions include, but are not necessarily limited to, calcium, cadmium, lithium, magnesium, cerium, cesium, chromium, cobalt, deuterium, gallium, iodine, iron, or zinc.

The positive ion can be provided in the form of any suitable salt or organic complex, including, but not necessarily limited to chloride, fluoride, hydroxide, phosphate, or sulfate salts. For example, where the positive ion is calcium, the ion can be in the form of calcium carbonate, calcium chloride, calcium fluoride, calcium hydroxide, calcium phosphates, or calcium sulfate.

The positive ion (e.g. calcium) can be provided in the composition of the invention at a concentration in the range of from about 10 umol to 10 mmol/ml, usually from about 50 umol to 5 mmol/ml, and more usually from about 100 umol to 1 mmol/ml. The term “umol” is used throughout to refer to micromole.

Where the positive ion in the adjuvant composition of the invention is calcium, it can be in combination with or substituted by other positive ions, including cadmium, lithium, magnesium, cerium, cesium, chromium, cobalt, deuterium, gallium, iodine, iron, and zinc, wherein the ions can be in the form of inorganic salts or organic complexes. The resulting composition is a PIC-containing adjuvant that further contains an antibiotic and a positive ion. In a particular embodiment, where the antibiotic is kanamycin and the ion is calcium the product may be described as PICKCa. In a related embodiment the PICKCa composition may contain molecules without restriction of different physical characteristics.

PIKA Adjuvant Composition

In an embodiment of particular interest, the polynucleotide adjuvant is PIKA. PIKA may be produced in a variety of ways, with production from PICKCa being of particular interest. PIKA may be produced from PICKCa through additional manufacturing processes that involves the isolation and/or concentration of molecules of a defined molecular size and/or weight. The separation and concentration of polynucleotide molecules of particular characteristics using filtration, chromatography, thermal treatment, centrifugal separation, electrophoresis, and similar methods that are standard processes and are known to those skilled in the art.

The immunogenic composition may be prepared as a dry powder, liquid solution, suspension or emulsion. The preparation of formulations of a desired immunogenic composition is generally described in Vaccine 4^{th} Edition by Stanley A Plotkin et al., W. B. Saunders Company; 4th edition 2003. Suitable formulations are also described in, e.g., A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.; Methods in Molecular Medicine, Vol. 87: Vaccine Protocols, 2nd edition (2003), Humana Press; Mucosal Vaccines (1996), Kiyono et al., eds., Academic Press; and Vaccine Adjuvants: Preparation Methods and Research Protocols (2000) D. T. O'Hagan, Humana Press.

In embodiments of particular interest, the invention features an adjuvant generally referred to as PIKA comprising a polyriboinosinic-polyribocytidylic acid (PIC), an antibiotic (e.g., kanamycin), and a positively charged ion (e.g., a calcium ion), wherein the composition contains molecules of the adjuvant heterogeneous for molecular weight having a molecular weight of from about 66,000 to 1,200,000 Daltons. That is, the adjuvant composition comprises molecules with a weight distribution in the range of from about 66,000 to 1,200,000 Daltons.

In related embodiments, the PIKA polynucleotide adjuvant composition molecules in the composition are heterogeneous, that is the weight of the adjuvant molecules are distributed within a range of molecular weight, where the molecular weight is from about 300,000 to 1,200,000 Daltons, or from about 66,000 to 660,000 Daltons, or from about 300,000 to 660,000 Daltons, or from about 300,000 to 2,000,000 Daltons, or from about 66,000 Daltons to about 100,000 Daltons, 100,000 to 200,000 Daltons, from about 300,000 Daltons to about 4,000,000 Daltons, or from about 500,000 Daltons to 1,000,000 Daltons, or from about 1,000,000 Daltons to 1,500,000 Daltons, or from about 1,500,000 Daltons to 2,000,000 Daltons, or from about 2,000,000 Daltons to 2,500,000 Daltons, or from about 2,500,000 Daltons to 3,000,000 Daltons, or from about 3,000,000 Daltons to 3,500,000 Daltons, or from about 3,500,000 Daltons to 4,000,000 Daltons, or from about 4,000,000 Daltons to 4,500,000 Daltons, or from about 4,500,000 Daltons to 5,000,000 Daltons.

In related embodiments, the PIKA polynucleotide adjuvant composition molecules in the composition have an average molecular weight or equal to or greater than 66,000 Daltons, equal or greater than 150,000 Daltons, or equal to or greater than 250,000 Daltons, or equal to or greater than 350,000 Daltons, or equal to or greater than 500,000 Daltons, or equal to or greater than 650,000 Daltons, or equal to or greater than 750,000 Daltons, or equal to or greater than 1,000,000 Daltons, or equal to or greater than 1,200,000 Daltons, or equal to or greater than 1,500,000 Daltons, or equal to or greater than 2,000,000 Daltons.

In embodiments of particular interest, the invention features an adjuvant generally referred to as PIKA comprising a polyriboinosinic-polyribocytidylic acid (PIC), an antibiotic, and a positive ion wherein the composition contains molecules of the adjuvant heterogeneous, that is the size of the adjuvant molecules are distributed within a range of molecular size, for molecular size having a sediment co-efficient Svedbergs (S) of from about 6.43S to 24.03S.

In related embodiments, the PIKA polynucleotide adjuvant composition molecules in the composition are heterogeneous, that is the size of the adjuvant molecules are distributed within a range of molecular size, where the molecular size is from about 12.8S to 24.03S, or from about 3S to 12S or from about 6.43 to 18.31S, or from about 12.8 to 18.31S, or from about 12.8S to 30.31S, or from about 12.8S to 41.54S, or from about 13.5S, to 18.31S, or from about 13.5S to 24.03S, or from about 16.14 to 22.12S, or from about 22.12S to 26.6S, or from about 26.6S to 30.31S, or from about 30.31S to 33.55S, or from about 33.55S to 36.45S, or from about 36.45S to 39.1S, or from about 39.1S to 41.54S, or from about 41.54S to 43.83S, or from about 43.83S to 45.95S.

In further related embodiments, the PIKA polynucleotide adjuvant composition has an average sedimentation co-efficient (Svedbergs) greater than 9, or greater than 12, or greater than 13.5, or greater than 15, or greater than 16, or greater than 17, or greater than 18, or greater than 19, or greater than 20, or greater than 21, or greater than 22 or greater than 25, or greater than 30.

Immunogenic Properties

An immunogenic composition, including PIKA and an antigen, can generally induce an antigen-specific immune response in at least two ways: i) humoral-mediated immunity, which includes B cell stimulation and production of antibodies or immunoglobulins (other cells are also involved in the generation of an antibody response, e.g. antigen-presenting cells, including macrophages and helper T cells (Th1 and Th2), and ii) cell-mediated immunity, which generally involves T cells including cytotoxic T lymphocytes, although other cells are also involved in the generation of a cytotoxic T lymphocyte response (e.g., Th1 and/or Th2 cells and antigen presenting cells).

Furthermore, the polynucleotide adjuvant composition may alter the quality of the immune response by affecting the subclasses (isotypes) of immunoglobulins produced, as well as their affinities.

The degree and nature of the immunogenic response induced by a subject immunogenic composition may be thus assessed by measuring the presence of molecules including cytokines, chemokines and antibodies produced by cells of the immune system.

The current invention provides for novel immunogenic substances comprising the PIKA adjuvant that enhance the overall level of immune response in a host by inducing a mucosal immune response. In certain embodiments, a subject immunogenic composition induces a mucosal immune response and enhances the systemic level of immunity. The induction of a mucosal immune response as well as the enhancement of the systemic immunity is of interest in treating infectious diseases caused by pathogenic organisms that enter the body through a mucosal surface.

The examples provided demonstrate that an immunogenic composition comprising PIKA and a SARS antigen, when administered by peritoneal injection induce systemic immune response, where the expression of specific IgA and specific IgG in the blood are measures of systemic immune activity. However, identical immunogenic composition comprising PIKA and a SARS antigen, when administered by peritoneal injection did not induce a mucosal immune response, where the expression of S-IgA is a measure of the mucosal immune activity.

Surprisingly, the identical immunogenic composition comprising PIKA and a SARS antigen, when administered mucosally induces a mucosal immune response, as indicated by the expression of specific S-IgA in the mucosal surface.

Example 1 illustrates that the presence of the PIKA adjuvant in an immunogenic composition administered by peritoneal injection does not induce an enhanced expression of specific S-IgA in the mucosal membrane. However, the presence of the PIKA adjuvant in an immunogenic composition administered mucosally induces the expression of specific S-IgA in the mucosal membrane in a dose dependent manner (Table A).

The presence of the PIKA adjuvant in an immunogenic composition administered by peritoneal injection elicited a dose dependent increase in the presence of IgA in the blood. Further, the presence of the PIKA adjuvant in the immunogenic composition administered mucosally also increased the level of specific IgA in the blood in a dose dependent manner (Table B).

Further, the presence of the PIKA adjuvant in an immunogenic composition administered by peritoneal injection elicited a dose dependent increase in the presence of IgG in the blood. The presence of the PIKA adjuvant in the immunogenic composition administered mucosally also increased the level of specific IgG in the blood in a dose dependent manner (Table C).

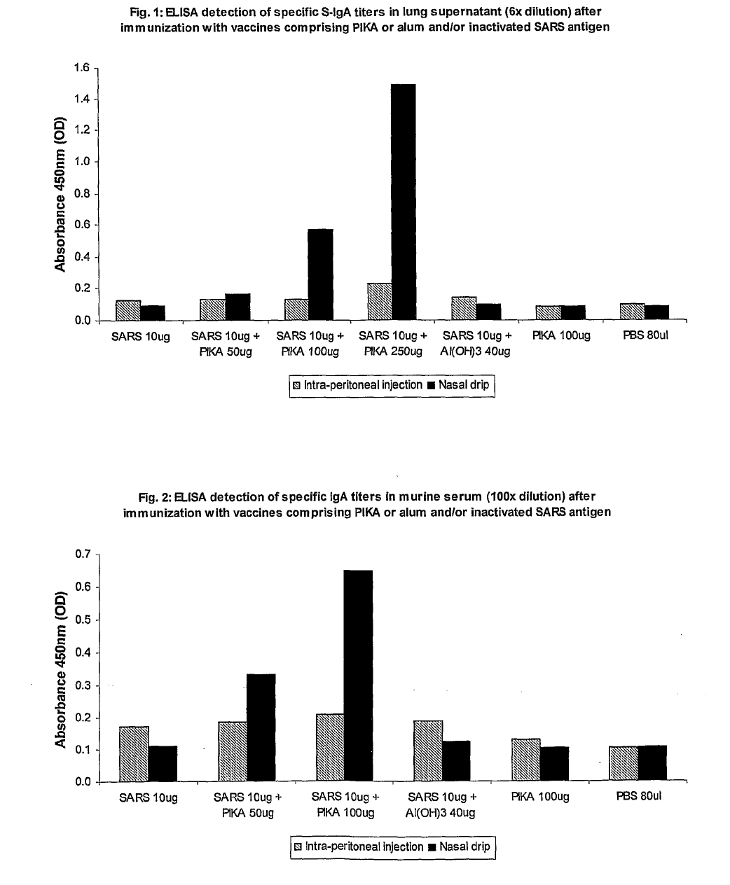

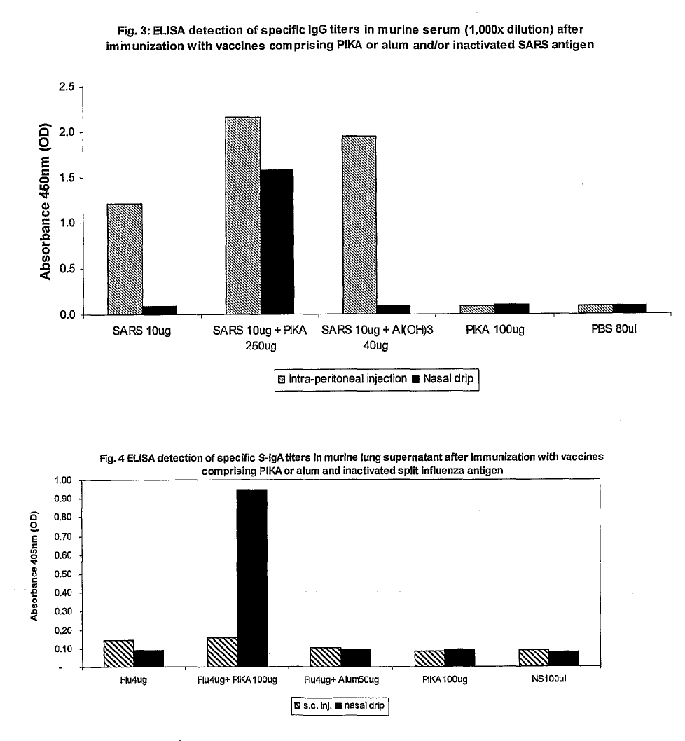

The results of these examples are summarized in FIGS. 1 to 3.

The production of specific IgG in the blood induced by mucosal delivery of the vaccine composition of PIKA and SARS antigen, was over to 70% of the observed levels for peritoneal delivery (Table B). Thus the presence of PIKA in an immunogenic substance delivered mucosally has the additional unexpected benefit of inducing an immune response in both the mucosal and systemic immune sub-systems.

Example 2 demonstrates that the presence of PIKA induce both a mucosal and systemic immune response. Further the mucosal administration of an immunogenic composition comprising PIKA was unexpectedly observed to induce a mucosal immune response at a remote mucosal site. In addition, the mucosal administration of an immunogenic composition comprising PIKA was unexpectedly observed to induce a T cell mediated immune response.

In Example 2 influenza antigen used was an approved human influenza vaccine VAXIGRIP from Sanofi Pasteur comprising, H1N1, H3N2 like strains and b/Shanghai5/361/2002 strain.

The influenza antigen alone and a composition comprising the influenza antigen plus PIKA administered by subcutaneous injection induces a strong specific systemic humoral immune response but only a weak specific mucosal immune response as measured by the production of S-IgA in the mucosal surfaces of the lung and intestine.

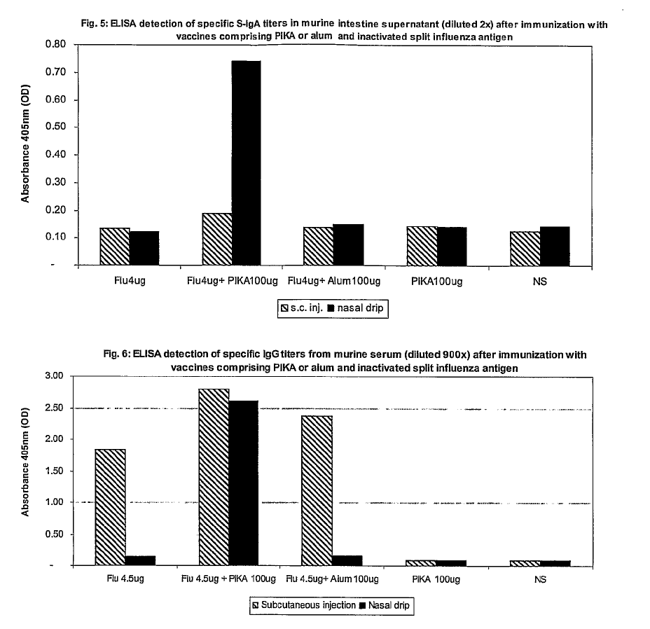

The administration of the influenza antigen alone and the influenza antigen combined with alum (a recognized vaccine antigen) via a nasal drip also induced only a weak specific mucosal immune response (see Tables E and F, FIGS. 4 and 5) as measured by the production of S-IgA in the mucosal surfaces of the lung and intestine.

In contrast, the presence of PIKA in an immunogenic composition comprising the influenza antigen induced an unexpectedly strong specific mucosal site in the mucosal surface of the lung as measured by the production of S-IgA (Table E FIG. 4)

Further it was observed that at the remote mucosal site of the intestine there was also a strong specific mucosal immune response as indicated by the presence of S-IgA (Table F FIG. 5)

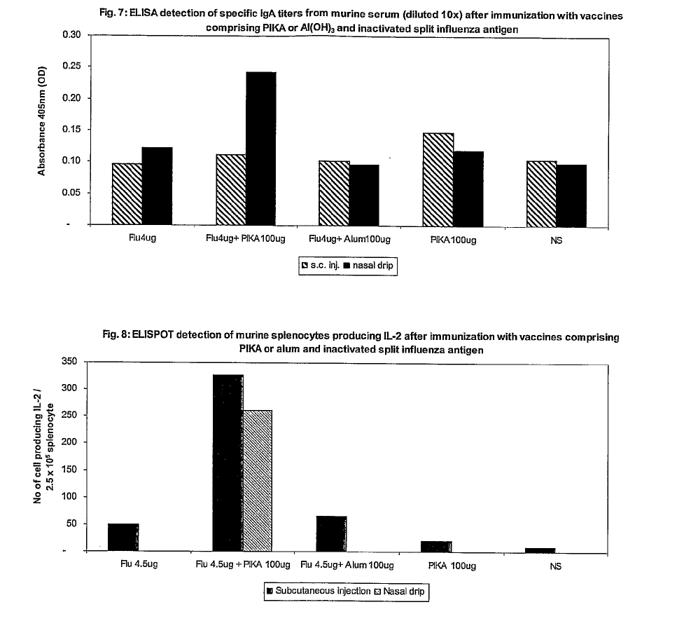

In addition the administration of an immunogenic composition comprising PIKA and the influenza antigen induced a strong specific systemic response both humoral as measured by specific IgA and specific IgG in the blood serum (see Tables G and H, FIGS. 6 and 7) as well as a T cell mediated immune response and measured by the production of I1-2 by splenocytes (Table I FIG. 8).

Example 3 further demonstrates that the presence of PIKA enhances the mucosal immune response while also specifically amplifying the cell mediated immune response. In comparison, the use of an alum adjuvant under identical experimental conditions did not enhance either the degree of mucosal immune activity or the cell mediated immune response.

Additional Features

In a further embodiment a subject immunogenic composition is farther defined by the relative presence of the PIKA adjuvant and the antigen or antigens where the presence is measured in terms of one or more characteristics of quantity, concentration, volume, number of molecules or other recognized metric.

In related embodiments, a subject immunogenic composition comprises a polynucleotide adjuvant composition and an antigen or antigens where the presence of the adjuvant and the antigen in terms of weight or number of molecules is in a ratio of less than 1 to 1,000, of less than 1 to 900, of less than 1 to 800, of less than 1 to 700, of less than 1 to 500, of less than 1 to 400, of less than 1 to 300, of less than 1 to 200, of less than 1 to 100, of less than 1 to 50, of less than 1 to 10, of less than 1 to 5, of less than 1 to 2, of about 1 to 1, of greater than 2 to 1, of greater than 5 to 1, of greater than 10 to 1, of greater than 50 to 1, of greater than 100 to 1, of greater than 200 to 1, of greater than 300 to 1, of greater than 400 to 1, of greater than 500 to 1, of greater than 600 to 1, of greater than 700 to 1, of greater than 800 to 1, of greater than 900 to 1, of greater than 1,000 to 1.

In a further related embodiment, a subject immunogenic composition is defined in terms of dose; that is the quantity of immunogenic composition that is to be administered to induce the optimal beneficial immune response or alternatively the range of dose that may be administered from the minimum required to elicit an immune response to the maximum dose beyond which the incremental beneficial response is not medically justified in the context of the potential inducement of adverse side effects.

In certain embodiments of particular interest, the immunogenic composition comprises a polynucleotide adjuvant composition and antigen where the presence of the antigen in a unit dose is provided in a quantity, that is more than 0.1 ug, is more than 0.5 ug is more than 0.001 mg is more than 0.005 mg, is more than 0.01 mg, is more than 0.025 mg, is more than 0.05 mg, is more than 0.075 mg, 0.1 mg is more than 0.25 mg, is more than 0.5 mg, is more than 1.2 mg, is more than 1.4 mg, is more than 1.6 mg, is more than 1.8 mg, is more than 2.0 mg is more than 2.5 mg, is more than 3 mg, is more than 3.5 mg, is more than 4 mg, is more than 5 mg, is more than 6 mg, is more than 7 mg, is more than 8 mg, is more than 9 mg, is more than 10 mg, is more than 15 mg, is more than 20 mg, is more than 25 mg, or is more than 50 mg

An optimal amount of antigen and the optimal ratio of antigen to PIKA adjuvant can be ascertained by standard studies involving observations of antibody titers and other immunogenic responses in the host.

Antigens

In an embodiment of particular interest the invention provides for an immunogenic composition comprising a polynucleotide adjuvant composition and an antigen or vaccine, where the source of the antigen is a human antigen, a non-human animal antigen, a plant antigen, one or more agents from infectious agents from any virus, bacteria including mycobacterium, fungus or parasite, cancer antigen, allergenic agents and other antigens, such as for developing autoimmune diseases.

In certain embodiments, the antigens may be derived from a natural source either crude or purified and used in its original live form or after having been killed, or inactivated, or truncated, or attenuated, or transformed into a non-reverting form, or detoxified, or mutated into a nontoxic form, or filtered or purified.

In some embodiments, the antigen is an isolated micro-organism antigen for example, a viral antigen, a bacterial antigen, a fungal antigen, an allergy antigen, a cancer antigen or an autoimmune antigen. In other embodiments, the antigen is a whole, inactivated antigen. Methods of inactivating a whole antigens are well known in the art; any known method can be used to inactivate an antigen and can be selected appropriately for the type of antigen of interest. Such methods of inactivating an antigen include for example, use of photoreactive compounds; oxidizing agents; irradiation (e.g., UV irradiation; γ-irradiation); combinations of riboflavin and UV irradiation; solvent-detergent treatment (e.g., treatment with organic solvent tri-N-butyl-phosphate with a detergent such as Tween 80); polyethylene glycol treatment; pasteurization (heat treatment); and low pH treatment; mild enzymatic treatment with pepsin or trypsin; Methylene blue (MB) phototreatment; treatment with Dimethylmethylene blue (DMMB) and visible light; treatment with S-59, a psoralen derivative and UVA illumination; and the like.

In a related embodiment of particular interest the antigen may be synthesized by means of solid phase synthesis, or may be obtained by means of recombinant genetics, or may be otherwise manufactured artificially so as to imitate the immunogenic properties of a pathogen.

The antigen may be acellular, capsular, infectious clone, replicon, vectored, microencapsulated, monovalent, bivalent or multivalent.

In some embodiments, a subject immunogenic composition comprises a polynucleotide adjuvant, and at least two different antigens, e.g., in some embodiments, a subject immunogenic composition comprises two antigens, three antigens, four antigens, five antigens, or more than five antigens.

Polypeptide antigens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. One may employ solid phase peptide synthesis techniques, where such techniques are known to those of skill in the art. See Jones, The Chemical Synthesis of Peptides (Clarendon Press, Oxford)(1994). Generally, in such methods a peptide is produced through the sequential additional of activated monomeric units to a solid phase bound growing peptide chain. Well-established recombinant DNA techniques can be employed for production of polypeptides, such methods include, but are not limited to, for example, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

In some embodiments, the antigen is a purified antigen, e.g., from about 25% to 50% pure, from about 50% to about 75% pure, from about 75% to about 85% pure, from about 85% to about 90% pure, from about 90% to about 95% pure, from about 95% to about 98% pure, from about 98% to about 99% pure, or greater than 99% pure.

The antigen may be acellular, capsular, infectious clone, replicon, vectored, microencapsulated, monovalent, bivalent or multivalent.

The polynucleotide adjuvant composition of the present invention can also be utilized to enhance the immune response against antigens produced by the use of DNA vaccines and/or DNA expressed proteins. The DNA sequences in these vaccines coding for the antigen can be either “naked” or contained in a delivery system, such as liposomes.

In one aspect of particular interest the novel vaccine composition may be defined by the selection of antigen or antigens that are used in combination with the PIKA adjuvant.

In an embodiment of particular interest, the present invention provides for a polynucleotide adjuvant composition and method of use where the polynucleotide adjuvant composition comprises the PIKA adjuvant together with an antigen wherein exemplary antigens include but are not limited to antigens that are of infectious disease pathogens which enter the host through a mucosal surface as described in Table N. Accordingly, Table N describes organisms that can serve as a source of antigens, and the diseases that can result following infection of the mucosal membrane.

TABLE N

| | |
|---|---|
| Pathogen Taxonomy | Disease |

Adenoviridae

Mastadenovirus

| | |
|---|---|
| Human adenovirus A to F | Common cold |

Arenaviridae

Old world arenaviruses

| Ippy virus | |
|---|---|
| Lassa virus | Lassa fever |
| Lymphocytic choriomeningitis virus | Lymphocytic choriomeningitis disease |

Astroviridae

Mamastrovirus

| | |
|---|---|
| Human astrovirus | Gastroenteritis |

Caliciviridae

Norovirus

| | |
|---|---|
| Norwalk virus | Diarrhea |

Flaviviridae

Hepadnaviridae

Orthohepadnavirus

| | |
|---|---|
| Hepatitis B virus | Hepatitis B |

| | |
|---|---|
| Hepatitis delta virus | Hepatitis D |
| Hepeviridae | |

Hepevirus

| | |
|---|---|
| Hepatitis E virus | Hepatitis E |

Herpesviridae

Alphaherpesvirinae

Simplexvirus

| | |
|---|---|
| Cercopithecine herpesvirus 1 | B Virus Infection |
| Human herpesvirus 1 | Herpes simplex type 1 |
| Human herpesvirus 2 | Herpes simplex type 2 |

Varicellovirus

| | |
|---|---|
| Human herpesvirus 3 (Varicella zoster virus) | Chicken pox, Shingels |

Betaherpesvirinae

Cytomegalovirus

| | |
|---|---|
| Human herpesvirus 5 | Cytomegalovirus (CMV) |

Gammaherpesvirinae

Lymphocryptovirus

| | |
|---|---|
| Human herpesvirus 4 | Epstein-Barr virus Infection |

Rhadinovirus

| | |
|---|---|
| Human herpesvirus 8 | Herpes |

Mononegavirales

Filoviridae

| Ebola-like viruses | |
|---|---|
| Ebola virus | Ebola disease |
| Marburgvirus | Marburg hemorrhagic fever |

Paramyxoviridae

Paramyxovirinae

Henipavirus

| | |
|---|---|
| Hendra virus | Hendra virus disease |

Morbillivirus

| | |
|---|---|
| Measles virus | Measles |

Respirovirus

| | |
|---|---|
| Human parainfluenza virus 1 | Human parainfluenza virus |
| Human parainfluenza virus 3 | Human parainfluenza virus |

Rubulavirus

| | |
|---|---|
| Human parainfluenza virus 2 | Human parainfluenza virus |
| Human parainfluenza virus 4 | Human parainfluenza virus |
| Mumps virus | Mumps |

Pneumovirinae

Metapneumovirus

| | |
|---|---|
| Human metapneumovirus | Human metapneumovirus |

Pneumovirus

| | |
|---|---|
| Human respiratory syncytial virus | Human respiratory syncytial disease |

Nidovirales

Coronaviridae

Coronavirus

Group 2 species

| | |
|---|---|
| Human coronavirus | Coronovirus |
| SARS coronavirus | SARS |

Torovirus

| | |
|---|---|
| Human torovirus | Torovirus disease |

Picornaviridae

Aphthovirus

| Equine rhinitis A virus | |
|---|---|
| Foot-and-mouth disease virus | Foot-and-mouth disease virus |

Enterovirus

Human enterovirus A

| | |
|---|---|
| Human coxsackievirus | Human coxsackievirus |
| Human enterovirus | Human enterovirus |

Human enterovirus B

| | |
|---|---|
| Enterovirus | Human enterovirus |
| Human coxsackievirus | Human coxsackievirus |
| Human echovirus | Human echovirus |

Human enterovirus C

| | |
|---|---|
| Human coxsackievirus | Human coxsackievirus |

Human enterovirus D

| | |
|---|---|
| Human enterovirus | Human enterovirus |

Poliovirus

| | |
|---|---|
| Human poliovirus | Polio |

| | |
|---|---|
| Human enterovirus sp. | Human enterovirus |
| unclassified Enteroviruses | |

| | |
|---|---|
| Human enterovirus sp. | Human enterovirus |

Hepatovirus

| | |
|---|---|
| Hepatitis A virus | Hepatitis A virus |

Parechovirus

| | |
|---|---|
| Human parechovirus | Human parechovirus |

Human parechovirus

Rhinovirus (common cold viruses)

Human rhinovirus A

| | |
|---|---|
| Human rhinovirus | Common cold |

Human rhinovirus B

| | |
|---|---|
| Human rhinovirus | Common cold |

unclassified Rhinovirus

| | |
|---|---|
| Human rhinovirus | Common cold |

Orthomyxoviridae

Influenzavirus A

| | |
|---|---|
| Influenza A virus | Influenza |

Influenzavirus B

| | |
|---|---|
| Influenza B virus | Influenza |

Influenzavirus C

| | |
|---|---|
| Influenza C virus | Influenza |

Paramyxoviridae

Paramyxovirinae

Henipavirus

| | |
|---|---|
| Hendra virus | Hendra virus |

Papillomaviridae

Alphapapillomavirus

| | |
|---|---|
| Human papillomavirus | Human papillomavirus |

Betapapillomavirus

| | |
|---|---|
| Human papillomavirus | Human papillomavirus |

Gammapapillomavirus

| | |
|---|---|
| Human papillomavirus | Human papillomavirus |

Mupapillomavirus

| | |
|---|---|
| Human papillomavirus | Human papillomavirus |

unclassified Papillomaviridae

| | |
|---|---|
| Human papillomavirus types | Human papillomavirus |

Parvoviridae

Parvovirinae

Erythrovirus

Human parvovirus
unclassified Erythrovirus

| | |
|---|---|
| Human erythrovirus | Human erythrovirus |

Polyomaviridae

Polyomavirus

| | |
|---|---|
| JC polyomavirus | Progressive multifocal leukencephalopathy |

Poxviridae

Chordopoxvirinae

Orthopoxvirus

| | |
|---|---|
| Variola virus | Smallpox |

Reoviridae

Rotavirus

| | |
|---|---|
| Rotavirus A | Diarrhea |
| Rotavirus B | Diarrhea |
| Rotavirus C | Diarrhea |

Retroviridae

Orthoretrovirinae

Deltaretrovirus

Primate T-lymphotropic virus 1

| | |
|---|---|
| Human T-lymphotropic virus 1 | Human T-lymphotropic virus |

Primate T-lymphotropic virus 2

| | |
|---|---|
| Human T-lymphotropic virus 2 | Human T-lymphotropic virus |

Primate T-lymphotropic virus 3

| | |
|---|---|
| Human T-lymphotropic virus 3 | Human T-lymphotropic virus |

Lentivirus

Primate lentivirus group

| | |
|---|---|
| Human immunodeficiency virus type 1 and 2 | HIV |

unclassified Retroviridae

Aids-associated retrovirus
Human endogenous retroviruses

Togaviridae

Alphavirus
Rubivirus

| | |
|---|---|
| Rubella virus | Rubella, German Measels |

Actinobacteria

Actinobacteria (class) (high G + C Gram-positive bacteria)

Acidimicrobidae

Actinobacteridae

Actinomycetales

Corynebacterineae

Corynebacteriaceae

Corynebacterium

| | |
|---|---|
| Corynebacterium diptheriae | Diphtheria |

Actinobacteridae

Actinomycetales

Corynebacterineae

Mycobacteriaceae

Mycobacterium

| | |
|---|---|
| Mycobacterium abscessus | Mycobacterium abscessus infection |

| | |
|---|---|
| Mycobacterium avium complex | Mycobacterium abscessus infection |
| Mycobacterium leprae | Leprosy/Hansen's Disease |
| Mycobacterium tuberculosis | Mycobacterium tuberculosis Infection |

Nocardiadeae

Nocardia

| | |
|---|---|
| Nocardia asteroides | Nocardiosis |
| Nocardia farcinica | Nocardiosis |
| Nocardia nova | Nocardiosis |
| Nocardia transvalensis | Nocardiosis |
| Nocardia brasiliensis | Nocardiosis |
| Nocardia pseudobrasiliensis | Nocardiosis |

Chlamydiae/Verrucomicrobia group

Chlamydiae

Chlamydiae (class)

Chlamydiales

Chlamydiaceae

Chlamydia

| | |
|---|---|
| Chlamydia trachomatis | Chlamydia |

| | |
|---|---|
| Chlamydia pneumoniae | Pneumonia |
| Chlamydia psittaci | Psittacosis |
| Chlamydia trachomatis, serovars A, B, Ba, and C | Trachoma |
| Chlamydophila pneumoniae | Pneumonia |

Firmicutes (Gram-positive bacteria)

Bacilli

Bacillales

Bacillaceae

Bacillus

Bacillus cereus group

| | |
|---|---|
| Bacillus anthracis | Anthrax |

Listeriaceae

Listeria

| | |
|---|---|
| Listeria monocvtogenes | Listeriosis |

Staphylococcaceae

Staphylococcus

| Staphylococcus aureus | Methicillin Resistant |
|---|---|
| | Staphylococcus aureus (MRSA) |
| Staphylococcus aureus VISA and VRSA | Staphylococcus |

aureus(VISA/VRSA) Infections

Lactobacillales

Streptococcaceae

| | |
|---|---|
| Streptococcus | Streptococcal Diseases |

| | |
|---|---|
| Group A streptococcus | Scarlet Fever |
| Group B streptococcus | Meningitis |
| Streptococcus pneumoniae | Pneumonia |

Clostridia

Clostridiales

Clostridaceae

Clostridium

| | |
|---|---|
| Clostridium botulinum | Botulism |
| Clostridium difficile | Diarrhea |

Mollicutes

Mycoplasmatales

Mycoplasmataceae

Mycoplasma

| | |
|---|---|
| Mycoplasma pneumonia | Mycoplasma pneumoniae Infection |

Proteobacteria (purple bacteria and relatives)

Alphaproteobacteria

Rhizobiales (rhizobacteria)

Brucellaceae

| | |
|---|---|
| Brucella | Brucellosis |

Betaproteobacteria

Burkholderiales

Alcaligenaceae

Bordetella

| | |
|---|---|
| Bordetella pertussis | Pertussis |

Burkholderiaceae

Burkholderia

Burkholderia cepacia complex

| | |
|---|---|
| Burkholderia cepacia | Burkholderia cepacia Infection |
| Burkholderia pseudomallei | Melioidosis |

Neisseriales

Neisseriaceae

Neisseria

| | |
|---|---|
| Neisseria gonorrhoeae | Gonorrhea |

| | |
|---|---|
| Neisseria meningitidis, meningococcus | Meningitis |

delta/epsilon subdivisions

Epsilonproteobacteria

Camplobacterales

Campylobacteraceae

| | |
|---|---|
| Campylobacter | Campylobacter Infection |

| | |
|---|---|
| Campylobacter jejuni | Diarrhea |

Helicobacteraceae

Heliobacter

| | |
|---|---|
| Heliobacter pylori | Helicobacter pylori Infection |

Gammaproteobacteria

Enterobacteriales

Entrobacteriaceae

Escherichia

| | |
|---|---|
| Escherichia coli | Dysentery |

| | |
|---|---|
| Salmonella | Salmonellosis |

| | |
|---|---|
| Salmonella typhi | Salmonella typhi Infection/Typhoid |

Shigella

| | |
|---|---|
| Shigella dysenteriae | Dysentery |
| Shigella flexneri | Diarrhea |
| Shigella sonnei | Shigellosis |

| | |
|---|---|
| Yersinia | Yersiniosis |

Legionellales

Coxiellaceae

Coxiella

| | |
|---|---|
| Coxiella burnetii | Q Fever |

Legionellaceae

Legionella

| | |
|---|---|
| Legionella pneumophila | Legionellosis/Legionnaire's Disease |
| Legionella pneumophila | Pontiac Fever |

Pasteurellales

Pasteurellaceae

Haemophilus

| | |
|---|---|
| Haemophilus ducreyi | Haemophilus ducreyi Infection |

| | |
|---|---|
| Haemophilus influenzae serotype b | Haemophilus influenzae |

Serotype b (Hib) Infection

Pseudomonadales

Pseudomonadaceae

Pseudomonas

Pseudomonas aeruginosa group

| | |
|---|---|
| Pseudomonas aeruginosa | Pseudomonas aeruginosa infection |

Vibrionales

Vibrionaceae

Vibrio

| | |
|---|---|
| Vibrio parahaemolyticus | Vibrio parahaemolyticus Infection |
| Vibrio vulnificus | Vibrio vulnificus Infection |
| Vibrio cholerae | Cholera |

Spirochaetes

Spirochaetes (class)

Spirochaetales

Leptospiraceae

| | |
|---|---|
| Leptospira | Leptospirosis |

Treponema

| | |
|---|---|
| Treponema pallidun | Syphilis |

Ascomycota (ascomycetes)

Pezizomycotina

Eurotiomycetes

Eurotiales

Trichocomaceae

mitosporic Trichocomaceae

| | |
|---|---|
| Aspergillus | Aspergillosis |

Onygenales

Ajellomycetaceae

Ajellomyces

Ajellomyces capsulatus

| | |
|---|---|
| Histoplasma capsulatum | Histoplasmosis |

| | |
|---|---|
| Blastomycoides dermatitidis | Blastomycosis |

mitosporic Onygenales

Coccidiodes

| | |
|---|---|
| Coccidiodes immitis | Coccidioidomycosis, Valley fever |

Paracoccidioides

Paracoccidioides brasiliensis

Pneumocystidomycetes

Pneumocystidales

Pneumocystidaceae

Pneumocystis

| | |
|---|---|
| Pneumocystis jiroveci | PCP Infection |

Saccharomycotina

Saccharomycetes

Saccharomycetales

mitosporic Saccharomycetales

Candida

| | |
|---|---|
| Candida albicans | Candidiasis, Thrush |

Basidiomycota (basidiomycetes)

Hymenomycetes

Heterobasidiomycetes

Tremellomycetidae

Tremellales

Tremellaceae

Filobasidiella

Filobasidiella neoformans

| | |
|---|---|
| Cryptococcus neoformans | Cryptococcosis |

Phylum Sarcomastigophora (the protozoa)

Subphylum Mastigophora (the flagellates)

Class Zoomastigophorea

Order Trichomonadida

| | |
|---|---|
| Dientamoeba fragilis | Dientamoeba fragilis |
| Dientamoeba fragilis | Infection |

Order Diplomonadida

| | |
|---|---|
| Giardia lamblia (giardiasis) | Giardiasis/Giardia |
| Giardia intestinalis | Infection |

Subphylum Sarcodina (the amoebae)

Superclass Rhizopoda

Class Lobosea

Order Amoebida

| | |
|---|---|
| Entamoeba histolytica | Amebiasis |
| (amoebiasis, amoebic dysentery) | |
| Entamoeba histolytica | |

Phylum Apicomplexa

Class Sporozoea

Subclass Coccidia

Order Eucoccidiorida

Suborder Eimeriorina

Family Eimeriina

| | |
|---|---|
| Isospora belli | Isospora Infection |
| Isospora belli | |

Family Sarcocystidae

| | |
|---|---|
| Toxoplasma gondii (toxoplasmosis) | Toxoplasmosis |
| Toxoplasma gondii | |

Family Cryptosporidiidae

| | |
|---|---|
| Cryptosporidium parvum (cryptosporidosis) | Cryptosporidiosis |
| Cryptosporidium | |
| Cyclospora cayetanesis | Cyclosporiasis |
| Cyclospora cayetanensis | |

Phylum Ciliophora (the ciliates)

Class Litostomatea

Order Vestibuliferida

| | |
|---|---|
| Balantidium coli | Balantidium Infection |
| Balantidium coli | |

Phylum Plathyhelminthes (the flatworms)

Class Trematoda

Subclass Digenea (the digenetic trematodes)

Order Echinostomatiformes

Family Fasciolidea

| | |
|---|---|
| Fasciolopsis buski | Fasciolopsiasis |

Order Opisthorchiformes

Family Heterophyidae

| | |
|---|---|
| Heterophyes heterophyes | Heterophyes Infection |

Phylum Nematoda (the roundworms)

Class Rhabditae

Order Strongylida

Family Ancylostomidae

| | |
|---|---|
| Angiostrongylus cantonensis | Angiostrongyliasis |

Order Ascaridida

| | |
|---|---|
| Ascaris spp. (human and pig roundworms) | Ascaris Infection |
| Anisakis simplex and Pseudoterranova decipiens | Anisakiasis |

Order Spirurida

Suborder Camallanina

Family Dracunculidae

| Dracunculus medinensis (guinea worm, fiery serpent) | |
|---|---|
| Dracunculus medinensis | Guinea Worm Disease |

In an embodiment of particular interest, the present invention provides for a polynucleotide adjuvant composition and method of use where the polynucleotide adjuvant composition comprises the PIKA adjuvant together with a allergy antigen that enters the host through a mucosal surface wherein the antigen is from a human or animal allergy source including; plants, animals, fungi, insects food, dust and mites and the like.

Allergens include but are not limited to environmental aeroallergens; plant pollens such as ragweed/hayfever; weed pollen allergens; grass pollen allergens; Johnson grass; tree pollen allergens; ryegrass; arachnid allergens, such as house dust mite allergens (e.g., Der p I, Der f I, etc.); storage mite allergens; Japanese cedar pollen/hay fever; mold spore allergens; animal allergens (e.g., dog, guinea pig, hamster, gerbil, rat, mouse, etc., allergens); food allergens (e.g., allergens of crustaceans; nuts, such as peanuts; citrus fruits); insect allergens; venoms: (Hymenoptera, yellow jacket, honey bee, wasp, hornet, fire ant); Other environmental insect allergens from cockroaches, fleas, mosquitoes, etc.; bacterial allergens such as streptococcal antigens; parasite allergens such as Ascaris antigen; viral antigens; fungal spores; drug allergens; antibiotics; penicillins and related compounds; other antibiotics; whole proteins such as hormones (insulin), enzymes (streptokinase); all drugs and their metabolites capable of acting as incomplete antigens or haptens; industrial chemicals and metabolites capable of acting as haptens and functioning as allergens (e.g., the acid anhydrides (such as trimellitic anhydride) and the isocyanates (such as toluene diisocyanate)); occupational allergens such as flour (e.g., allergens causing Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Altemaria (Altemaria altemata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella gennanica); Apis (e.g. Apis multiflorum); Cupressus (e.g. Cupressus sempervirens, Cupressus arizonica and Cupressus macroarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherun (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In an embodiment of particular interest, the present invention provides for a polynucleotide adjuvant composition and method of use where the polynucleotide adjuvant composition comprises the PIKA adjuvant together with an autoimmune antigen that enters the host through a mucosal surface.

Additional Agents

In some embodiments, a subject immunogenic composition comprises, in addition to a polynucleotide adjuvant and an antigen, one or more additional agents, e.g., immunomodulatory agents, carriers, and the like.

In an embodiment of particular interest, the present invention provides for an immunogenic composition and method of use, where the immunogenic composition comprises the PIKA adjuvant, an antigen or vaccine together with another immunomodulating substance, including adjuvants, where suitable immunomodulating substances include, but are not limited to: an aluminum composition such as aluminum hydroxide; oil-in-water emulsions compositions or emulsions comprising an immunogenic substances, including Complete Freund's Adjuvant; an oil-in-water emulsion containing dried, heat-killed Mycobacterium tuberculosis organisms; Incomplete Freund's Adjuvant; emulsions including mycobacterial cell wall components; emulsions including squalene (MF-59); detoxified endotoxins, lipid A derivatives including monophosphoryl lipid A-microbial (MPL); baptens; nitrocellulose-absorbed protein; saponins including particulate immunomodulators isolated from the barck of Quillaja Saponoria for example QS21; endogenous human immunomodulators; bacterial derived adjuvants including umnethylated CpG dinucleotides; oligodeoxynucleotides (e.g., synthetic oligonucleotides) containing unmethylated CpG dinucleotides; liposomes (e.g., liposomes comprising biodegradable materials such as phospholipids); (e.g., microspheres made from a variety of polymers such as polylactic-co-glycolic acid (PLGA), polyphosphazene and polyanhydrides); Interlukin-2; Bacillus Calmette Guerin; Granulocyte Monocyte-Colony Stimulating Factor; Montanide ISA-51; Keyhole limpet hemocyanin; DNA; proteins; encapsulated antigens; and immune stimulating complexes (ISCOM's); cholera toxin, choleral toxin derivatives; zonula occludens toxin; escherichia coli heat-labile enterotoxin; labile toxin, labile toxin derivatives; pertussis toxin, pertussis toxin derivatives; muramyl dipeptide derivatives; seppic series of montanide adjuvants; poly-di(carboxylatophenoky)phosphazene and leislunania elongation factor.

When the subject immunogenic composition is administered in conjunction with another adjuvant, the polynucleotide adjuvant can be administered before and/or after, and/or simultaneously with the other adjuvant. For example the polynucleotide adjuvant may be administered with the initial administration of the antigen, followed by a boost dose of vaccine comprising either or both of the adjuvants. Alternatively the initial dose of vaccine administered may exclude the polynucleotide adjuvants but an immunogenic substance comprising the polynucleotide adjuvant is subsequently administered to the patient.

In certain embodiments the subject immunogenic composition may be administered with cytokines or other co-stimulatory molecules for example: IL-1, IL-2, IL-4, IL-5, IL-6, IL-7, IL-10, IL-12, IL-15

In a related embodiment the present invention provides for an immunogenic substance comprising the PIKA adjuvant, an antigenic substance or substances plus a suitable carrier. The carrier may be for example an oil-and-water emulsion, a lipid vehicle or aluminum salt, cochleates, ISCOMs, liposomes, live bacterial vectors, live viral vectors, microspheres, nucleic acid vaccines, polymers, polymer rings, sodium fluoride, transgenic plants, virosomes, virus like particles, and other delivery vehicles known in the art.

The polynucleotide adjuvant may be directly administered to the subject or may be administered in conjunction with a delivery complex. Where the delivery complex is a substance associated with a targeting means e.g. a molecule that results in higher affinity binding to target cell such as dendritic cell surfaces and/or increased cellular uptake by target cells. Examples of delivery complexes include but are not limited to; nucleic acid delivery acids associated with: a sterol (e.g. cholesterol), a lipid (e.g. cationic lipid, virosome or liposome), or a target cell specific binding agent (e.g. a ligand recognized by a target cell specific receptor). Preferred complexes may be sufficiently stable in vivo to prevent significant uncoupling prior to internalization by the target cell. However, the complex may be cleavable under appropriate conditions within the cell.

In one embodiment of interest, the composition comprising PIKA adjuvant does not include poly-L-lysine or a derivative thereof.

Kits

In certain embodiments, the invention provides a kit comprising a subject immunogenic composition. In certain embodiments, the invention provides a kit comprising a polynucleotide adjuvant and an antigen in separate formulations.

In certain embodiments, the invention provides for a kit comprising the polynucleotide adjuvant and an immunogenic compound.

In a related embodiment, the invention provides for a kit comprising the polynucleotide adjuvant and an immunogenic compound where the immunogenic substance is an antigen.

In some embodiments, a subject kit comprises a subject immunogenic composition in a sterile liquid (e.g., aqueous) formulation, where the formulation is sterile, and is provided in a sterile container, a sterile vial, or a sterile syringe.

In some embodiments, a subject kit comprises a subject immunogenic composition formulated for injection. In some embodiments, a subject kit comprises a subject immunogenic composition in a sterile liquid formulation, contained within a sterile syringe; and a needle. In some embodiments, a subject kit comprises a subject immunogenic composition in a sterile liquid formulation in a unit dosage amount (e.g., a single dose), contained within a sterile syringe; and a needle.

In some embodiments, a subject kit comprises a subject immunogenic composition, lyophilized and in a sterile container; and a container comprising a sterile liquid for reconstitution of the lyophilized composition. In some embodiments, the kit further comprises instructions for reconstitution of the lyophilized composition.

In some embodiments a subject kit comprises an immunogenic composition formulated for administration rectally, vaginally, nasally, orally (including inhalation), opthanialically, topically, pulmonary, ocularly or transdermally and an appropriate delivery device for example, inhaler, suppository, applicator or the like,

A subject kit in some embodiments will further include instructions for use, including e.g., dosage amounts and dosage frequencies. Instructions are in some embodiments printed directly on the kit. In other embodiments, instructions are printed material provided as a package insert. Instructions can also be provided in other media, e.g., electronically in digital or analog form, e.g., on an audio cassette, an audio tape, a compact disc, a digital versatile disk, and the like.

Formulations

A subject immunogenic composition is provided in any of a variety of formulations. For example, a subject immunogenic composition may be prepared as an injectable, dry power, liquid solution, for example: aqueous or saline solutions, suspension, cream, emulsion, tablet, pill, dragee, capsule, gel, syrup or slurry. In some embodiments, a subject immunogenic composition is formulated for mucosal delivery: e.g., delivery via inhalation, delivery via the respiratory tract, oral delivery, rectal delivery, vaginal delivery, etc. The preparation of formulations of a desired immunogenic composition is generally described in Vaccine 4^{th} Edition by Stanley A Plotkin et al., W. B. Saunders Company; 4th edition 2003. Suitable formulations are also described in, e.g., A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A subject immunogenic composition may be microencapsulated, encochleated, coated onto microscopic gold partiles, contained in liposomes, nebulized aerosols, pellets for implantation into the skin, or dried onto a sharp object to be scratched into the skin.

In a further embodiment the subject immunogenic substance may be delivered alone or in conjunction with a dispersion system. In some embodiments the dispersion system is selected from the group consisting of for example: macromolecular complexes, nanocapsules, microspheres, beads and lipid based systems. Lipid based systems optionally include oil-in-water emulsions, micelles, mixed micelles or liposomes.

In certain embodiments a subject immunogenic composition comprising the PIKA adjuvant is in the form of a pharmaceutically acceptable solution, which may routinely contain pharmaceutically acceptable concentrations of salt, buffering agents, preservatives, compatible carriers, adjuvants and optionally other therapeutic ingredients. The composition may contain additives for example: disintegrants, binders, coating agents, swelling agents, lubricants, flavorings, sweeteners or solubilizers and the like.

In certain embodiments a subject immunogenic composition comprising the PIKA adjuvant is administered in its neat for or in the form of a pharmaceutically acceptable salt.

In certain embodiments, the PIKA adjuvant composition and an immunogenic composition comprising the PIKA adjuvant and antigenic compound may be freeze-dried (lyophilized) for long term stability and storage in a solid form. The freeze-dried method is lnown to those skilled in the art.

In one aspect of particular interest, the invention provides for an adjuvant composition or immunogenic composition wherein the immunogenic composition, or the adjuvant composition contained in the immunogenic composition, is in a solid or liquid form or in solution or in suspension.

For parenteral administration in an aqueous solution, for example, the solution should be suitably buffered if necessary and the liquid diluent first rendered isotonic with sufficient saline or glucose. These particular aqueous solutions are especially suitable for intravenous and intraperitoneal administration. In this connection, sterile aqueous media which can be employed will be known to those of skill in the art in light of the present disclosure. Exemplary injection media which can be used in the present invention include a buffer with or without dispersing agents and/or preservatives, and edible oil, mineral oil, cod liver oil, squalene, mono-, di- or triglyceride, and a mixture thereof.

A subject immunogenic composition will in some embodiments be formulated in specific forms suitable for mucosal administration. Such forms, both sterile and non-sterile, may include for example; capsules, liquid solutions, liquid drops, emulsions, suspensions, elixirs, creams, suppositories, gels, capsules including soft capsules, sprays, inhalants, aerosols, powders, tablets, coated tablets, microcapsules, suppositories, drops, pills, dragees, syrups, slurries, enemas, granules, or lozenges. Any inert carrier can be used, such as saline, or phosphate buffered saline, stabilizers, propellants, encased in a gelatin capsule or a microcapsule or vector which aids mucosal application or any such carrier in which the compounds used in the method of the present invention have suitable solubility properties for use in the methods of the present invention.

A subject immunogenic composition may be administered to an individual by means of a pharmaceutical delivery system for the inhalation route (oral, intratracheal, intranasal). Thus, a subject immunogenic composition may be formulated in a form suitable for administration by inhalation. The pharmaceutical delivery system is one that is suitable for respiratory therapy by topical administration of a subject bacterial composition to mucosal linings of the bronchi. This invention can utilize a system that depends on the power of a compressed gas to expel the bacteria from a container. An aerosol or pressurized package can be employed for this purpose. Thus, in some embodiments, a subject immunogenic composition is formulated for delivery to a respiratory tissue, e.g., by inhalation. In some embodiments, a subject immunogenic composition is aerosolized to create an aerosol.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains the immunogenic composition, which can be dissolved, suspended, or emulsified in a mixture of a fluid carrier and a propellant. In some embodiments, a subject immunogenic composition is formulated with a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a subject. Various types of propellants known to one of skill in the art can be utilized. Examples of suitable propellants include, but are not limited to, hydrocarbons or other suitable gas. In the case of the pressurized aerosol, the dosage unit may be determined by providing a value to deliver a metered amount.

There are several different types of inhalation methodologies which can be employed in connection with the present invention. A subject immunogenic composition can be formulated in basically three different types of formulations for inhalation. First, a subject immunogenic composition can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the subject as discussed within U.S. Pat. Nos. 5,404,871 and 5,542,410.

Alternatively, a subject immunogenic composition can be formulated in aqueous or ethanolic solutions and delivered by conventional nebulizers. In some embodiments, such solution formulations are aerosolized using devices and systems such as disclosed within U.S. Pat. Nos. 5,497,763; 5,544,646; 5,718,222; and 5,660,166.

Furthermore, a subject immunogenic composition can be formulated into dry powder formulations. Such formulations can be administered by simply inhaling the dry powder formulation after creating an aerosol mist of the powder. Technology for carrying such out is described within U.S. Pat. No. 5,775,320 and U.S. Pat. No. 5,740,794. Formulations suitable for intranasal administration include nasal sprays, nasal drops, aerosol formulations; and the like.

The present invention provides a package for use in delivering a subject immunogenic composition into an airway or respiratory tract of an individual. In general, a package suitable for delivery into a respiratory tract comprises a container that holds a flowable formulation suitable for delivery to the respiratory tract (e.g., by inhalation), a polynucleotide adjuvant as described above, and an antigen. In some embodiments, the package is a metered dose inhaler, and the polynucleotide adjuvant and the antigen are formulated with a propellant.

In some embodiments, a subject immunogenic composition is formulated as a sustained release formulation (e.g. a controlled release formulation). For example, in some embodiments, a subject immunogenic composition is formulated into pellets or cylinders and implanted intramuscularly or subcutaneously as depot injections or as implants. Such implants will generally employ known inert materials such as biodegradable polymers. Injectable depot forms are made by forming microencapsule matrices of a subject immunogenic composition in biodegradable polymers such as polylactide-polyglycolide. Examples of other suitable biodegradable polymers include poly(orthoesters) and poly(anhydrides). Depot injectable formulations are also prepared by entrapping the composition in liposomes or microemulsions which are compatible with body tissue. Delivery release systems also include the following examples: polymer based systems, microcapsules, lipids, hydrogel release systems, sylastic systems, peptide systems, peptide based systems, wax coatings, compressed tablets, partially fused implants, Other forms of sustained release are known by those skilled in the art.

For oral delivery, a subject immunogenic composition will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, a subject immunogenic composition is formulated together with one or more pharmaceutical excipients and coated with an enteric coating, as described in U.S. Pat. No. 6,346,269. For example, a subject immunogenic composition and a stabilizer are coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, tale, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methaerylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate(HPMCP), cellulose acetate phthalate (CAP), polyvinyl phthalic acetate (PVPA), Eudragit™ and shellac.

Suitable oral formulations also include a subject immunogenic composition formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B. V.).

Suitable oral formulations also include a subject immunogenic composition formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like. Also suitable for use are formulations such as those described in U.S. Pat. No. 6,296,842 (Alkermes Controlled Therapeutics, Inc.); U.S. Pat. No. 6,187,330 (Scios, Inc.); and the like

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

In a related embodiment, a subject immunogenic composition is formulated with one or more agents that inhibit degradation by gastrointestinal enzymes and/or acids. In some embodiments, a subject immunogenic composition is formulated with one or more agents that protect the components of the composition from degradation by gastrointestinal enzymes and/or acids.

In some embodiments, a subject immunogenic composition is formulated with one or more agents that enhance absorption by mucosal tissues.

In some embodiments, a subject immunogenic composition is formulated for vaginal delivery, providing a vaginal delivery system. In one exemplary embodiment, the vaginal delivery system is a tampon or tampon-like device that comprises a subject immunogenic composition. Drug delivery tampons are known in the art, and any such tampon can be used in conjunction with a subject drug delivery system. Drug delivery tampons are described in, e.g., U.S. Pat. No. 6,086,909 If a tampon or tampon-like device is used, there are numerous methods by which subject immunogenic composition can be incorporated into the device. For example, the subject immunogenic composition can be incorporated into a gel-like bioadhesive reservoir in the tip of the device. Alternatively, the subject immunogenic composition can be in the form of a powdered material positioned at the tip of the tampon. The subject immunogenic composition can also be absorbed into fibers at the tip of the tampon, for example, by dissolving the subject immunogenic composition in a pharmaceutically acceptable carrier and absorbing the subject immunogenic composition into the tampon fibers. The subject immunogenic composition can also be dissolved in a coating material which is applied to the tip of the tampon. Alternatively, the subject immunogenic composition can be incorporated into an insertable suppository which is placed in association with the tip of the tampon.

In other embodiments, a subject immunogenic composition is formulated for use with a vaginal ring, providing vaginal delivery system that is a vaginal ring. Vaginal rings usually consist of an inert elastomer ring coated by another layer of elastomer containing a subject immunogenic composition. The rings can be easily inserted, left in place for the desired period of time (e.g., up to 7 days), then removed by the user. The ring can optionally include a third, outer, rate-controlling elastomer layer which contains no immunogenic composition. The subject immunogenic composition can be incorporated into polyethylene glycol throughout the silicone elastomer ring to act as a reservoir for the subject immunogenic composition.

In other embodiments, a suitable vaginal delivery system is a vaginal sponge. The subject immunogenic composition is incorporated into a silicone matrix which is coated onto a cylindrical drug-free polyurethane vaginal sponge, as described in the literature. Pessaries, tablets and suppositories are other examples of drug delivery systems which can be used in the present invention. These systems have been described extensively in the literature.

Another system is a container comprising a subject immunogenic composition (e.g., a tube) that is adapted for use with an applicator for, e.g., rectal or vaginal delivery. A subject immunogenic composition is incorporated into creams, lotions, foams, paste, ointments, and gels which can be applied to the vagina using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

Methods

In one aspect of particular interest, the invention provides for a method for eliciting and/or enhancing immune responses to an antigenic compound, comprising administering to a host a subject immunogenic composition. In some embodiments, the host is a human. In other embodiments, the host is a non-human animal, e.g., a non-human mammal, an avian species, etc.

Furthermore, the present invention provides a method for enhancing immune responses to an antigenic compound by administering to a host the immunogenic composition. The host can be a human being or non-human animal. The administration can be delivered parenterally by injection, such as intramuscular, intraperitoneal, intravenous, subcutaneous or intradermal injection. In other embodiments the immunogenic composition may be administered intradermally in ways other than by injection, for example, without breaching the epithelial barrier by mechanical means. In other embodiments, the immunogenic composition can be delivered rectally, vaginally, nasally, orally (including inhalation), opthamalically, topically, pulmonary, ocularly or transdermally.

The subject may be exposed to the antigen through environmental contact and therefore at risk of developing for example, an allergic reaction, an infectious disease, autoimmune disease or a cancer. In other embodiments the subject has for example an infectious disease, autoimmune disease, a cancer or allergy as a result of prior exposure to an antigen through environmental contact.

In certain embodiments the adjuvant is administered together with the antigen. In further embodiments the adjuvant is administered prior to or post the administration of the antigen.

A subject immunogenic composition will in some embodiments be administered via mucosal administration. Mucosal administration includes administration to the respiratory tissue, e.g., by inhalation, nasal drops, ocular drop, etc.; oral administration; anal or vaginal routes of administration, e.g., by suppositories; and the like.

In one aspect of particular interest, the invention provides for a method for enhancing immune responses to an antigenic compound, comprising administering to a host an immunogenic composition for enhancing the antigenicity of an antigenic compound comprising the polynucleotide adjuvant composition. In some of these embodiments, host is human. In other embodiments, the host is a non-human animal (e.g., a non-human primate, a rodent or other non-human mammal, an avian species, etc.)

In certain embodiments, the polynucleotide adjuvant composition can be used in the context of a vaccine. Optionally, the vaccine composition contains additional adjuvants. Vaccines classes included are anti-infectious respiratory, digestive, genitourinary or sensory diseases, allergy, and anti-autoimmune diseases.

A subject immunogenic composition is administered in an “effective amount” that is, an amount of a subject immunogenic composition that is effective in a selected route of administration to elicit, induce, or enhance an immune response. In some embodiments, an immune response is elicited to antigens produced by a pathogenic microorganism. In some embodiments, the amount of a subject immunogenic composition is effective to limit an infection, and/or to eradicate an infection, and/or to reduce a symptom associated with infection, by a pathogenic organism.

For example, in some embodiments, administration of a subject immunogenic composition to an individual is effective to treat an infectious disease, where treating an infectious disease, encompasses one or more of reducing the number of pathogenic agents in the individual (e.g., reducing viral load, reducing bacterial load, reducing the number of protozoa, reducing the number of helminths) and/or reducing a parameter associated with the infectious disease, including, but not limited to, reduction of a level of a product produced by the infectious agent (e.g., a toxin, an antigen, and the like); and reducing an undesired physiological response to the infectious agent (e.g., fever, tissue edema, and the like).

The exact amount of a subject immunogenic composition required to induce and/or enhance an immune response (e.g., a mucosal immune response) will vary from subject to subject, depending on the species, age, weight, and general conditions of the subject, the severity of the disease, infection, or condition that is being treated or prevented, the particular compound used, its mode administration, and the like. An appropriate amount may be determined by one of ordinary skill in the art using only routine experimentation given the teachings herein. Following an initial administration, subjects may receive one or several booster immunizations adequately spaced.

In some embodiments, serial doses of a subject immunogenic composition are administered. In these embodiments, the first dose of a subject immunogenic composition may be as a result of administering a vaccine. The second dose of a subject immunogenic composition is administered to the individual after the individual has been immunologically primed by exposure to the first dose. The booster may be administered days, weeks or months after the initial immunization, depending upon the patient's response and condition. For example, the booster dose is administered from about 2 days to about 12 months after the initial dose, e.g., from about 2 days to about 7 days, from about 1 week to about 2 weeks, from about 2 weeks to about 4 weeks, from about 4 weeks to about 8 weeks, from about 8 weeks to about 6 months, or from about 6 months to about 12 months after the initial dose. The present invention farther contemplates the use of a third, fourth, fifth, sixth or subsequent booster immunization, using, e.g., a third, fourth, fifth, sixth, or subsequent dose.

In certain embodiments the means of administration may comprise a combination of alternative routes, for example: systemically administered dose (e.g. peritoneal, inta-muscular, subcutaneous or intradermal administration) may be followed by mucosally delivered dose (e.g. intranasal, inhalation) or vice versa. At least one of the doses administered as part of the overall protocol would comprise the PIKA adjuvant.

In certain embodiments the polynucleotide adjuvant may be administered with either the first dose of antigen administered or any of the subsequent doses administered or all doses administered to the patient.

In certain embodiments the composition of the administered immunogenic composition may vary between the original administration and the boost and/or between booster doses. By way of an example the original dose administered may comprise a DNA vaccine while the booster dose is in the form of a recombinant protein vaccine. At least one of the doses administered as part of the overall protocol would comprise the PIKA adjuvant.

Whether an antibody response to an antigen has been induced or enhanced in an individual is readily determined using standard assays. For example, immunological assays such as enzyme-linked immunosorbent assays (ELISA), radioimmunoassay (RIA), immunoprecipitation assays, and protein blot (“Western” blot) assays; and neutralization assays (e.g., neutralization of viral infectivity in an in vitro or in vivo assay); can be used to detect the presence of antibody specific for a microbial antigen in a bodily fluid or other biological sample, e.g., the serum, secretion, or other fluid, of an individual.

Whether a CD4 immune response to an antigen has been induced in an individual is readily determined using standard assays, e.g., fluorescence-activated cell sorting (FACS) (see, e.g., Waldrop et al. (1997) J. Clin. Invest. 99:1739-1750); intracellular cytokine assays that detect production of cytokines following antigen stimulation (see, e.g., Suni et al. (1998) J. linmunol. Methods 212:89-98; Nomura et al. (2000) Cytometty 40:60-68; Ghanekar et al. (2001) Clin. Diagnostic Lab. Immunol. 8:628-631); MHC-peptide multimer staining assays, e.g., use of detectably labeled (e.g., fluorescently labeled) soluble MHC Class II/peptide multimers (see, e.g., Bill and Kotzin (2002) Arthritis Res. 4:261-265; Altman et al. (1996) Science 274:94-96; and Murali-Krishna et al. (1998) Immunity 8:177-187); enzyme-linked immunospot (ELISPOT) assays (see, e.g., Hutchings et al. (1989) J. Immunol. Methods 120:1-8; and Czerkinsky et al. (1983) J. Immunol. Methods 65:109-121); and the like. As one non-limiting example of an intracellular cytokine assay, whole blood is stimulated with antigen and co-stimulating antibodies (e.g., anti-CD28, anti-CD49d) for 2 hours or more; Brefeldin A is added to inhibit cytokine secretion; and the cells are processed for FACS analysis, using fluorescently labeled antibodies to CD4 and to cytokines such as TNF-a, IFN-γand IL-2.

Whether an antigen-specific CD8 (e.g., cytotoxic T cell; “CTL”) response is induced to an antigen (e.g., to a pathogen) can be determined using any of a number of assays known in the art, including, but not limited to, measuring specific lysis by CTL of target cells expressing the antigen on their surface, which target cells have incorporated a detectable label which is released from target cells upon lysis, and can be measured, using, e.g., a ⁵¹Cr-release assay; a lanthanide fluorescence-based cytolysis assay; and the like.

Subjects Suitable for Treatment

Subjects suitable for treatment with a subject method of inducing an immune response to a microbial pathogen, and methods of treating or preventing an infection with a microbial pathogen, include individuals who have been infected with a pathogenic microorganism; individuals who are susceptible to infection by a pathogenic microorganism, but who have not yet been infected; and individuals who are at risk of becoming infected with a pathogenic microorganism, but who have not yet been infected. Suitable subjects include infants, children, adolescents, and adults.

Subjects suitable for treatment with a subject method of inducing an immune response to a microbial pathogen, and methods of treating or limiting an infection with a microbial pathogen, include pediatric target population, e.g., individuals between about 1 year of age and about 17 years of age, including infants (e.g., from about 1 month old to about 1 year old); children (e.g., from about 1 year old to about 12 years old); and adolescents (e.g., from about 13 years old to about 17 years old).

Subjects suitable for treatment with a subject method of inducing an immune response to a microbial pathogen, and methods of treating or limiting an infection with a microbial pathogen, include neonates, e.g., an individual (e.g., a human neonate) from one day to about 14 days old, e.g., from about 1 day to about 2 days old, from about two days to about 10 days old, or from about 10 days to about 14 days old.

In a particular embodiment, the subject is a human child about ten years or younger, e.g., about five years old or younger, and the immunogenic compositions are administered at any one or more of the following times: two weeks, one month, 2 months, 3 months, 4 months, 5 months, 6 months, 7 months, 8 months, 9 months, 10 months, 11 months, 12 months, 15 months, 18 months, or 21 months after birth, or at 2 years, 3 years, 4 years, 5 years, 6 years, 7 years, 8 years, 9 years, or 10 years of age. In some embodiments, a subject immunogenic composition is administered to an individual in the age range of from about 6 months to about 6 years, where the individual receives a first dose at about 6 months of age, and subsequent booster doses, e.g., 2-3 subsequent booster doses, at, e.g., 2 years of age, 4 years of age, and 6 years of age.

In a particular embodiment, the subject is a human adult from about 17 years old to 49 years old. In some embodiments, the subject is an elderly human adult from 50 to 65 years old, 65 to 75 years old, 75 to 85 years old or over 85 years old.

In some embodiments, a subject immunogenic composition is administered to an individual shortly after contact (e.g., shortly after confirmed or suspected contact) with an actual or potential source of the microbial pathogen, for example, an individual who is known to have or suspected to have an infection with a microbial pathogen. For example, in some embodiments, a subject immunogenic composition is administered to an individual within about 1 hour, within about 2 hours, within about 5 hours, within about 8 hours, within about 12 hours, within about 18 hours, within about 24 hours, within about 2 days, within about 4 days, within about 7 days, within about 2 weeks, or within about one month after contact with an individual who is known to have or suspected to have an infection with a microbial pathogen.

In some embodiments, a subject immunogenic composition is administered to an individual that is known or may be suspected of being a carrier or a microbial pathogen whether or not they are showing symptoms of the infection.

Subjects suitable for treatment with a subject method of inducing an immune response to a microbial pathogen, and methods of treating or limiting an infection with a microbial pathogen, include CD4⁺ T cell-deficient individuals (“CD4⁺-deficient” individuals), e.g., individuals who have lower than normal numbers of fimctional CD4⁺ T lymphocytes. As used herein, the term “normal individual” refers to an individual having CD4⁺ T lymphocyte levels and function(s) within the normal range in the population, for humans, typically 600 to 1500 CD4⁺ T lymphocytes per mm³ blood. CD4⁺-deficient individuals include individuals who have an acquired immunodeficiency, or a primary immunodeficiency. An acquired immunodeficiency may be a temporary CD4⁺ deficiency, such as one caused by radiation therapy, or chemotherapy.

Also suitable for treatment with the methods of the invention are individuals with healthy, intact immune systems, but who are at risk for becoming CD4⁺ deficient (“at-risk” individuals). At-risk individuals include, but are not limited to, individuals who have a greater likelihood than the general population of becoming CD4⁺ deficient. Individuals at risk for becoming CD4⁺ deficient include, but are not limited to, individuals at risk for HIV infection due to sexual activity with HIV-infected individuals; intravenous drug users; individuals who may have been exposed to HIV-infected blood, blood products, or other HIV-contaminated body fluids; a baby who has passed through the birth canal of an HIV-infected individual; babies who are being nursed by HIV-infected mothers; and the like.

Subjects suitable for treatment with the formulations and methods of the instant invention for treating allergy include any individual who has been diagnosed as having an allergy. Subjects amenable to treatment using the methods and agents described herein include individuals who are known to have allergic hypersensitivity to one or more allergens. Subjects amenable to treatment include those who have any of the above-mentioned allergic disorders. Also amenable to treatment are subjects that are at risk of having an allergic reaction to one or more allergens. Also suitable are individuals who failed treatment with one or more standard therapies for treating an allergic disorder.

Subjects suitable for treatment include individuals living in industrialized nations; individuals living developing countries; individuals living in rural areas; individuals living in relatively isolated areas; and the like.

The target population for a subject immunogenic composition will vary, depending on the microbial pathogen

The above disclosure generally describes the present invention. The following examples will be of assistance to the understanding of the present invention. These examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Examples

### Example 1

### Systemic Immune Response Induced by the Peritoneal and Mucosal Administration of PIKA in Combination with a SARS Antigen

This example demonstrates that an immunogenic substance comprising PIKA and a SARS antigen induces a strong systemic immune response when administered by peritoneal injection and a strong immune response both at local and remote sites of administration, e.g., both a mucosal and a systemic immune response are elicited when administered mucosally.

Six groups of three balb/c mice were inoculated with a composition of SARS antigen plus the PIKA adjuvant (a heterogeneous composition of PIKA molecules predominantly within a weight range distribution of about 66 kDa to 1,200 kDa). The amount of antigen and adjuvant used is described in tables A to C below. A repeat inoculation was administered after two weeks and a further booster administered after a further two weeks.

In week six a blood sample was taken and the presence of specific IgA and specific IgG in the blood serum was detected by ELISA. The mice were sacrificed, the lungs were extracted, dissected and washed to draw out the supernatant. The resultant mucosal extract was tested for the presence of specific S-IgA.

The findings as presented in tables A, B and C (also FIGS. 1, 2 and 3) demonstrate that the presence of PIKA in the immunogenic composition administered by intra-peritoneal injection enhances the systemic immune response as measured by the dose dependent increase in expression of specific IgG in the blood. However, there was no observed impact on the mucosal immune activity as measured by the presence of specific S-IgA in the samples taken from the lungs. The presence of PIKA in the immunogenic composition administered mucosally enhances the mucosal immune response as measured by the dose dependent increase expression of specific S-IgA in the mucosal surfaces of the lungs. Further there was a dose dependent enhancement of systemic immune response as measured by the presence of specific IgA and IgG in the blood serum samples.

TABLE A
ELISA detection of specific IgA antibody titers in murine lung supernatant (diluted 6×) after
immunization with vaccines comprising PIKA or alum and/or whole inactivated SARS antigen

| | Groups of mice | | | | | | |
|---|---|---|---|---|---|---|---|
| | | Group 2 | Group 3 | Group 4 | Group 5 | | |
| | Group 1 | SARS 10 ug + | SARS 10 ug + | SARS 10 ug + | SARS 10 ug + | Group 6 | Group 7 |
| Administration | SARS 10 ug | PIKA 50 ug | PIKA 100 ug | PIKA 250 ug | Al(OH)3 40 ug | PIKA 100 ug | PBS 80 ul |
| Intra-peritoneal injection | 0.122 | 0.130 | 0.129 | 0.229 | 0.142 | 0.084 | 0.100 |
| Nasal drip | 0.089 | 0.163 | 0.570 | 1.485 | 0.095 | 0.088 | 0.087 |

Units: average optical density absorption 405 nm

TABLE B
ELISA detection of specific IgA antibody titers in murine serum (diluted 100×) after
immunization with vaccines comprising PIKA or alum and/or whole inactivated SARS antigen

| | | SARS 10 ug + | SARS 10 ug + | SARS 10 ug + | | |
|---|---|---|---|---|---|---|
| Route of Administration | SARS 10 ug | PIKA 50 ug | PIKA 100 ug | Al(OH)₃ 40 ug | PIKA 100 ug | PBS 80 ul |
| Intra-peritoneal injection | 0.171 | 0.183 | 0.205 | 0.186 | 0.129 | 0.104 |
| Nasal drip | 0.109 | 0.331 | 0.646 | 0.121 | 0.103 | 0.106 |

Units: average optical density absorption 405 nm

TABLE C
ELISA detection of specific IgG antibody titers in murine serum (diluted 1,000×) after
immunization with vaccines comprising PIKA or alum and/or whole inactivated SARS antigen

| | Groups of mice | | | | |
|---|---|---|---|---|---|
| | | Group 2 | Group 3 | | |
| | Group 1 | SARS 10 ug + | SARS 10 ug + | Group 4 | Group 5 |
| Administration | SARS 10 ug | PIKA 250 ug | Al(OH)3 40 ug | PIKA 100 ug | PBS 80 ul |
| Intra-peritoneal injection | 1.208 | 2.157 | 1.938 | 0.097 | 0.094 |
| Nasal drip | 0.091 | 1.574 | 0.092 | 0.098 | 0.096 |

Units: average optical density absorption 405 nm

### Example 2

### Mucosal and Systemic Immune Response Induced by the Administration of PIKA in Combination with an Influenza Antigen

This example demonstrates that an immunogenic substance comprising PIKA and an influenza antigen induces a strong mucosal immune response at both local and remote sites of administration i.e. at both the respiratory and intestinal mucosal membranes as well as a systemic immune response when administered mucosally.

Five groups of balb/c mice were vaccinated on day 0 and day 20 with compositions as described in table D.

TABLE D
Vaccine Composition and Administration Route

| | Mice | | | Route of |
|---|---|---|---|---|
| Group | per Group | Adjuvant | Antigen | Immunization |
| A | 4 | PIKA 100 ug | VAXIGRIP | Intra-nasal |
| | | | 4.5 ug | |
| B | 3 | | VAXIGRIP | Intra-nasal |
| | | | 4.5 ug | |
| C | 3 | Alum 50 ug | VAXIGRIP | Intra-nasal |
| | | | 4.5 ug | |
| D | 3 | PIKA 100 ug | | Intra-nasal |

| | | | |
|---|---|---|---|
| E | 3 | Neutral Saline Solution | Intra-nasal |

The influenza antigen used is an inactivated purified split influenza vaccine VAXIGRIP from Sanofi Pasteur that is approved for human use comprising, H1N1, H3N2 like strains and b/Shanghai5/361/2002 strain.

The samples of blood were collected after day 35 and tested for the presence of a specific humoral immune response in ELISA.

The mice were sacrificed after 7 weeks, the lungs and intestines were extracted, dissected and washed to draw out the supernatant. The resultant mucosal extract was tested for the presence of specific S-IgA in ELISA.

The findings as presented in table E demonstrate that the presence of PIKA in the immunogenic composition administered mucosally enhances the mucosal immune response in the lungs as measured by the expression of specific S-IgA in the mucosal surfaces of the lungs.

TABLE E
ELISA detection of specific S-IgA titers from murine lung
supernatant after immunization with vaccines comprising
PIKA and/or inactivated split influenza antigen

| | Groups of mice | | | | |
|---|---|---|---|---|---|
| | | Group 2 | Group 3 | | |
| | Group 1 | Flu 4.5 ug + | Flu 4.5 ug + | Group 4 | Group 5 |
| Administration | Flu 4.5 ug | PIKA 100 ug | Alum 100 ug | PIKA 100 ug | NS |
| Subcutaneous injection | 0.144 | 0.159 | 0.105 | 0.085 | 0.090 |
| Nasal drip | 0.091 | 0.947 | 0.094 | 0.095 | 0.081 |

Units: average optical density absorption 405 nm, NS: Neutral saline solution

Further, the findings as presented in table F (FIG. 5) demonstrates that presence of PIKA in the immunogenic composition administered mucosally enhances the mucosal immune response in the remote mucosal site of the intestine as measured by the expression of specific S-IgA in the mucosal surfaces of the intestine.

TABLE F
ELISA detection of specific S-IgA titers from murine intestine
supernatant after immunization with vaccines comprising
PIKA and/or inactivated split influenza antigen

| | Groups of mice | | | | |
|---|---|---|---|---|---|
| | | Group 2 | Group 3 | | |
| | Group 1 | Flu 4.5 ug + | Flu 4.5 ug + | Group 4 | Group 5 |
| Administration | Flu 4.5 ug | PIKA 100 ug | Alum 100 ug | PIKA 100 ug | NS |
| Subcutaneous injection | 0.133 | 0.190 | 0.137 | 0.144 | 0.124 |
| Nasal drip | 0.123 | 0.741 | 0.150 | 0.140 | 0.142 |

Units: average optical density absorption 405 nm

In addition, the findings presented below demonstrates that presence of PIKA in the immunogenic composition administered mucosally enhances the systemic immune response as measured by the expression of specific IgG (Table G, FIG. 6) and specific IgA (Table H, FIG. 7) in blood serum samples.

TABLE G
ELISA detection of specific IgG titers from murine blood serum after immunization
with vaccines comprising PIKA and/or inactivated split influenza antigen

| | Groups of mice | | | | |
|---|---|---|---|---|---|
| | | Group 2 | Group 3 | | |
| | Group 1 | Flu 4.5 ug + | Flu 4.5 ug + | Group 4 | Group 5 |
| Administration | Flu 4.5 ug | PIKA 100 ug | Alum 100 ug | PIKA 100 ug | NS |
| Subcutaneous injection | 1.839 | 2.804 | 2.371 | 0.087 | 0.089 |
| Nasal drip | 0.146 | 2.619 | 0.159 | 0.095 | 0.092 |

Units: average optical density absorption 405 nm

TABLE H
ELISA detection of specific IgA titers from murine blood serum after immunization
with vaccines comprising PIKA and/or inactivated split influenza antigen

| | Groups of mice | | | | |
|---|---|---|---|---|---|
| | | Group 2 | Group 3 | | |
| | Group 1 | Flu 4.5 ug + | Flu 4.5 ug + | Group 4 | Group 5 |
| Administration | Flu 4.5 ug | PIKA 100 ug | Alum 100 ug | PIKA 100 ug | NS |
| Subcutaneous injection | 0.096 | 0.112 | 0.102 | 0.147 | 0.104 |
| Nasal drip | 0.122 | 0.242 | 0.096 | 0.119 | 0.099 |

Units: average optical density absorption 405 nm

A suspension of spleen cells was prepared and a sample of the cell suspension from each mouse was put into 6-12 wells of the ELISPOT plate and cultured, Each well of the ELISPOT plate contained 200ul of splenocyte suspension, equivalent to approximately 2.5×10⁵ cells/well. For each mouse's sample of cultured splenocytes, half of wells containing the splenocytes were incubated with culture medium and the other half of wells were stimulated using the influenza antigen. Plates are incubated at 37° C. for 20 hours in environmentally controlled conditions prior to final preparation and reading using a standard ELISPOT plate reader.

Table I below (see also FIG. 7) presents the results for the number of cells per well producing IL-2. The administration of the immunogenic substance comprising PIKA and the influenza antigen was observed to induce a significantly higher level of IL-2 producing cells as compared with PIKA or the influenza antigen alone. This is indicates that the antigen with PIKA induces a T cell mediated immune response

TABLE I
ELISPOT detection of murine splenocytes producing IL-2 after immunization
with PIKA and/or inactivated split influenza antigen

| | Groups of mice | | | | |
|---|---|---|---|---|---|
| | | Group 2 | Group 3 | | |
| | Group 1 | Flu 4.5 ug + | Flu 4.5 ug + | Group 4 | Group 5 |
| Administration | Flu 4.5 ug | PIKA 100 ug | Alum 100 ug | PIKA 100 ug | NS |
| Subcutaneous injection | 49 | 327 | 65 | 20 | 10 |
| Nasal drip | | 262 | | | |

Units: average no. of cells producing Il-2 per 2.5 × 10⁵ splenocytes

### Example 3

### Mucosal and Systemic Immune Response Induced by the Administration of PIKA in Combination with an Influenza Antigen

This example demonstrates that an immunogenic substance comprising PIKA and an influenza antigen induces a strong antigen specific mucosal and systemic humoral immune response and T cell immune response after their administration to the mucosal surface.

Five groups of balb/c mice (three per group) were immunized on day 0, day 14 and day 30 with compositions as described in the tables below. The influenza antigen used is an inactivated purified split influenza vaccine VAXIGRIP from Sanofi Pasteur that is approved for human use comprising, H1N1, H3N2 like strains and b/Shanghai5/361/2002 strain.

The samples of blood were collected 14 days after the third immunization and tested for the presence of a specific serum IgG with ELISA.

The mice were sacrificed 14 days after the third immunization, the lungs and intestines were extracted, dissected and washed to draw out the supernatant. The resultant supernatant was tested for the presence of specific S-IgA in ELISA.

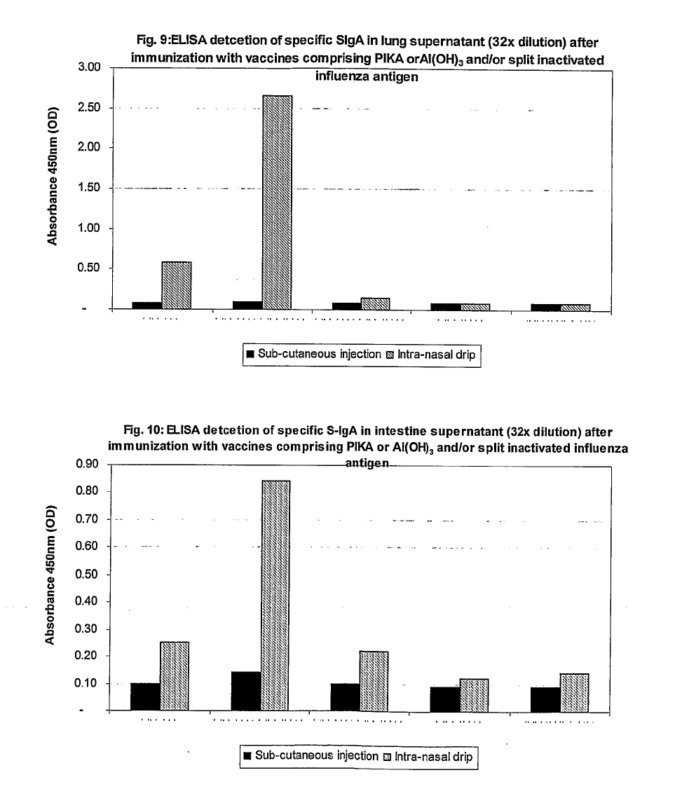

The findings as presented in table J (FIG. 9) demonstrate that the presence of PIKA in the immunogenic composition administered mucosally enhances the mucosal immune response in the lungs as measured by the expression of specific S-IgA in the mucosal surfaces of the lungs. The immunization with Al(OH)₃ and antigen intra-nasally did not induce production of S-IgA in the mucosal surface of lung.

TABLE J
ELISA detection of specific S-IgA in lung supernatant (32× dilution)
after immunization with vaccines comprising PIKA or Al(OH)₃ adjuvant
and/or split inactivated influenza

| | Mice groups | | | | |
|---|---|---|---|---|---|
| | | Flu 4.0 ug + | Flu 4.0 ug + | | Injectable |
| | Flu 4.0 ug | PIKA 100 ug | Alum 100 ug | PIKA 100 ug | Water |
| Subcutaneous injection | 0.08 | 0.09 | 0.08 | 0.08 | 0.08 |
| Intra-nasal drip | 0.59 | 2.66 | 0.15 | 0.08 | 0.08 |

Units: average optical density value

The findings as presented in table K (FIG. 10) demonstrate that the presence of PIKA in the immunogenic composition administered mucosally enhances the mucosal immune response in the intestine as measured by the expression of specific S-IgA in the mucosal surfaces of the intestine. The immunization of Al(OH)₃ with antigen intra-nasally did not induce production of S-IgA in the mucosal surface of intestine.

TABLE K
ELISA detection of specific S-IgA in intestine supernatant (32× dilution)
after immunization with vaccines comprising PIKA or Al(OH)₃ adjuvant
and/or split inactivated influenza antigen

| | Mice groups | | | | |
|---|---|---|---|---|---|
| | | Flu 4.0 ug + | Flu 4.0 ug + | | Injectable |
| | Flu 4.0 ug | PIKA 100 ug | Alum 100 ug | PIKA 100 ug | Water |
| Subcutaneous injection | 0.1 | 0.14 | 0.1 | 0.09 | 0.09 |
| Intra-nasal drip | 0.25 | 0.84 | 0.22 | 0.12 | 0.14 |

Units: average optical density value

A suspension of spleen cells was prepared and a sample of the cell suspension from each mouse was put into 6 wells of the ELISPOT plate and cultured, each well of the ELISPOT plate contained 200 ul of splenocyte suspension, equivalent to approximately 3.0×10⁵ cells/well. For each mouse's sample of cultured splenocyte, half of wells containing the splenocyte were incubated with culture medium and the other half of wells were stimulated using the influenza antigen. Plates are incubated at 37° C., 5% CO₂ for 20 hours prior to final preparation and reading using a standard ELISPOT plate reader.

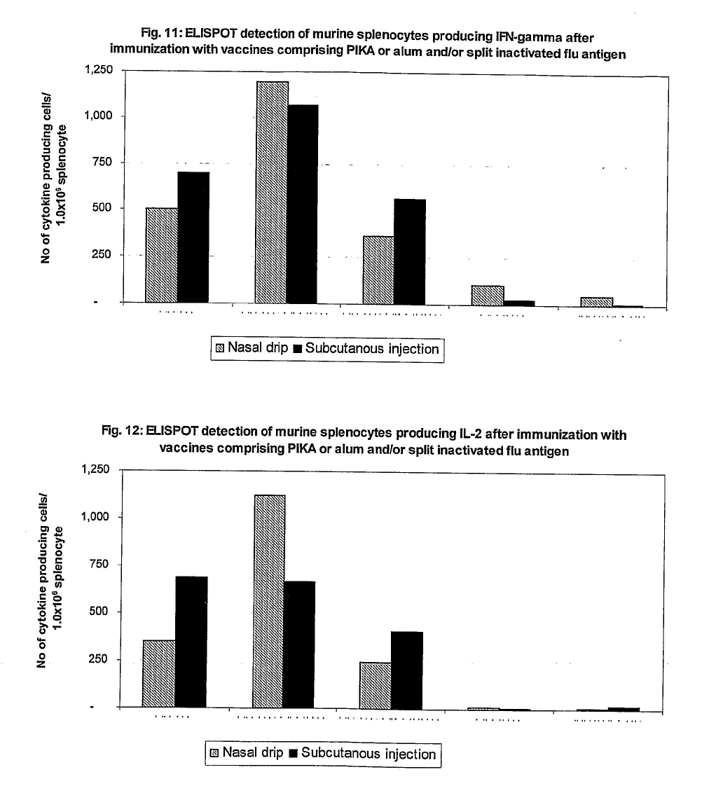

Table L below (see also FIG. 11) presents the results for the number of cells per 1.0×10⁶ splenocyte producing interferon-γ. The administration of the immunogenic substance comprising PIKA and the influenza antigen was observed to induce a significantly higher level of interferon-γ producing cells as compared with PIKA or the influenza antigen alone.

TABLE L
ELISPOT detection of murine splenocytes producing interferon-γ after
immunization with PIKA and/or inactivated split influenza antigen

| | | Flu 4.0 ug + | Flu 4.0 ug + | | Injectable |
|---|---|---|---|---|---|
| | Flu 4.0 ug | PIKA 100 ug | Al(OH)3 100 ug | PIKA 100 ug | Water |
| Nasal drip | 504 | 1,193 | 361 | 107 | 48 |
| Subcutanous | 700 | 1,068 | 566 | 28 | 8 |
| injection | | | | | |

Units: No. of cells producing interferon-γ per 1.0 × 10⁶ splenocytes

Table M below (see also FIG. 12) presents the results for the number of cells per 1.0×10⁶ splenocyte producing IL-2. The administration of the immunogenic substance comprising PIKA and the influenza antigen was observed to induce a significantly higher level of IL-2 producing cells as compared with PIKA or the influenza antigen alone.

TABLE M
ELISPOT detection of murine splenocytes producing interferon-γ after
immunization with PIKA and/or inactivated split influenza antigen

| | Mice Group | | | | |
|---|---|---|---|---|---|
| | | Flu 4.0 ug + | Flu 4.0 ug + | | Injectable |
| | Flu 4.0 ug | PIKA 100 ug | Al(OH)3 100 ug | PIKA 100 ug | Water |
| Nasal drip | 354 | 1,119 | 247 | 10 | 7 |
| Subcutanous | 687 | 663 | 406 | 8 | 17 |
| injection | | | | | |

Units: No. of cells producing Il-2 per 1.0 × 10⁶ splenocytes

The ability of PIKA to induce an amplified production of interferon-γ and IL-2 by splenocytes indicates that the immunization of antigen with PIKA intra-nasally and by subcutaneous injection induces a strong T cell mediated immune response. Immunization with Al(OH)₃ and antigen intra-nasally does not promote T cell response than just antigen alone.

However, the addition of Al(OH)₃ to the antigen does not promote an enhanced T cell immune response when administered intra-nasally or by subcutaneous injection.

1. An immunogenic composition comprising: (a) a polynucleotide adjuvant comprising: a polyriboinosinic-polyribocytidylic acid (PIC), at least one antibiotic, and at least one positive ion; and (b) at least one antigen; wherein the composition is formulated for mucosal administration.

2. The immunogenic composition according to claim 1, wherein the composition comprises polynucleotide adjuvant composition molecules heterogeneous for molecular weight, wherein the molecular weight is at least 66,000 Daltons.

3. The immunogenic composition according to claim 1 or claim 2, wherein the composition comprises polynucleotide adjuvant composition molecules heterogeneous for molecular weight, wherein the molecular weight is from about 66,000 to 1,200,000 Daltons.

4. The immunogenic composition according to any one of claims 1 to 3, wherein the composition comprises polynucleotide adjuvant composition molecules heterogeneous for molecular weight, wherein the molecular weight is at least 150,000 Daltons.

5. The immunogenic composition according to any one of claims 1 to 4, the immunogenic composition further comprising at least one immunomodulator.

6. The immunogenic composition according to any one of claims 1 to 5, wherein the immunogenic composition further comprises at least one agent that enhances mucosal absorption.

7. The immunogenic composition according to any one of claims 1 to 6, wherein the immunogenic composition or the adjuvant comprised in the immunogenic composition is in the form of a liquid, liquid solution, liquid drops, a solid, capsules, emulsions, suspensions, elixirs, creams, suppositories, gels, soft capsules, sprays, inhalants, aerosols, tablets, coated tablets, pill, dragee, powders, syrup, slurry, microcapsules, enemas, granules or lozenges.

8. The immunogenic composition according to any one of claims 1 to 7, wherein at least one of the adjuvant composition or the immunogenic composition is freeze-dried.

9. The immunogenic composition according to any one of claims 1 to 8, wherein the immunogenic composition is administered by inhalation, rectal delivery, vaginal delivery, nasal delivery, oral delivery, pulmonary delivery, ophthalmic delivery, topical delivery, ocular delivery or transdermal delivery.

10. The immunogenic composition according to any one of claims 1 to 9 for use in enhancing the mucosal immunogenic response of a host.

11. Use of the immunogenic composition, or the adjuvant comprised in the immunogenic composition, according to any one of claims 1 to 10 in the preparation of a medicament for enhancing the mucosal immunogenic response of a host.

12. The use according to claim 11, wherein the medicament is for enhancing a mucosal immune response at a local and remote site.

13. Use of the immunogenic composition, or the adjuvant comprised in the immunogenic composition, according to any one of claims 1 to 10 in the preparation of a medicament for inducing a T cell mediated immune response of a host.

14. The use according to any one of claims 11 to 13, wherein the medicament is administered by inhalation, rectal delivery, vaginal delivery, nasal delivery, oral delivery, pulmonary delivery, ophthalmic delivery, topical delivery, ocular delivery or transdermal delivery.

15. The use according to any one of claims 11 to 14, wherein the host has an infectious disease and by administering the antigenic compound elicits an immune response against the pathogen causing the infectious disease.

16. A kit comprising the immunogenic composition according to any one of claims 1 to 10.

17. A delivery system comprising an immunogenic composition according to any one of claims 1 to 10, wherein the delivery system enhances the delivery of the immunogenic composition to the mucosal surface.

18. A method for enhancing a mucosal immune response comprising administering to a host an immunogenic composition according to any one of claims 1 to 10.

19. The method according to claim 18, wherein the host has an infectious disease and the administering of the antigenic compound elicits an immune response against the pathogen causing the infectious disease.

20. A method for enhancing a mucosal immune response at a local and remote site comprising administering to a host a immunogenic composition according to any one of claims 1 to 10.

21. A method of inducing a T cell mediated immune response comprising administering to a host a immunogenic composition according to any one of claims 1 to 10.

22. The use according to any one of claims 11 to 15 or the method according to any one of claims 18 to 21, wherein the host is human.

23. The use according to any one of claims 11 to 15 or the method according to any one of claims 18 to 21, wherein the host is a non-human animal.
